(19) United States
(12) Patent Application Publication
(10) Pub. No.: US 2024/0400701 A1
(43) Pub. Date: Dec. 5, 2024

(54) ANTI-AMHRII ANTIBODIES

(71) Applicant: LANOVA MEDICINES DEVELOPMENT CO., LTD., Shanghai (CN)

(72) Inventors: Runsheng LI, Shanghai (CN); Wentao HUANG, Shanghai (CN); Yifan LI, Shanghai (CN)

(21) Appl. No.: 18/699,499

(22) PCT Filed: Oct. 8, 2022
(86) PCT No.: PCT/CN2022/123781

§ 371 Date: Apr. 8, 2024

(30) Foreign Application Priority Data
Oct. 8, 2021 (WO) PCT/CN2021/122601

(51) Int. Cl. C07K 16/28; A61P 35/00

(52) U.S. Cl. CPC C07K 16/2869; A61P 35/00; C07K 2317/24; C07K 2317/31; C07K 2317/565; C07K 2317/92

(57) ABSTRACT

Provided are anti-AMHRII antibodies and antigen-binding fragments, as well as their bispecific or multispecific counterparts. Methods of using the antibodies for treating and diagnosing diseases such as cancer are also provided.

### BACKGROUND

AMHRII (anti-Mullerian hormone receptor type 2, or AMHR2) is the receptor for the anti-Mullerian hormone (AMH) which, in addition to testosterone, plays an important role in male sex differentiation. AMH and testosterone are produced in the testes by different cells and have different effects. Testosterone promotes the development of male genitalia while the binding of AMH to the encoded receptor prevents the development of the mullerian ducts into uterus and Fallopian tubes. Mutations in this gene are associated with persistent Mullerian duct syndrome type II.

AMHRII is a serine/threonine kinase receptor homologous to type II receptors of the transforming growth factor-beta (TGF) superfamily. The AMH/AMHRII signaling also plays an important role in the regulation of oocyte development, and control of ovarian reserve and fertility in adult females.

It has been observed that AMHRII is overexpressed in the majority of human epithelial ovarian carcinoma (EOC). EOC is the most prevalent and lethal form of ovarian cancer representing about 85% of all ovarian cancers. Targeting the AMHRII with a therapeutic antibody, therefore, can be useful in treating cancers such as gynecologic and colorectal cancers.

### SUMMARY

Provided are anti-human AMHRII antibodies and fragments which are useful for treating diseases such as cancer characterized with expression or overexpression of the AMHRII protein.

In accordance with one embodiment of the present disclosure, provided is an antibody or antigen-binding fragment thereof comprising a heavy chain variable region (VH) comprising a VH CDR1, VH CDR2, and VH CDR3, and a light chain variable region (VL) comprising a VL CDR1, VL CDR2 and VL CDR3, wherein the VH CDR1 includes the amino acid sequence of SEQ ID NO:70; the VH CDR2 includes the amino acid sequence of SEQ ID NO:71; the VH CDR3 includes the amino acid sequence of SEQ ID NO:72; the VL CDR1 includes the amino acid sequence of SEQ ID NO:73; the VL CDR2 includes the amino acid sequence of SEQ ID NO:74; and the VL CDR3 includes the amino acid sequence of SEQ ID NO:18.

In some embodiments, the VH CDR1 includes the amino acid sequence of SEQ ID NO:13 or 20; the VH CDR2 includes the amino acid sequence of SEQ ID NO:14, 19, 21 or 33; the VH CDR3 includes the amino acid sequence of SEQ ID NO:15 or 22; the VL CDR1 includes the amino acid sequence of SEQ ID NO:16 or 23; the VL CDR2 includes the amino acid sequence of SEQ ID NO:17 or 24; and the VL CDR3 includes the amino acid sequence of SEQ ID NO: 18.

Also provided, in another embodiment, is an antibody or antigen-binding fragment thereof comprising a VH CDR1, VH CDR2, and VH CDR3, and a light chain variable region (VL) comprising a VL CDR1, VL CDR2 and VL CDR3, wherein the VH CDR1 includes the amino acid sequence of SEQ ID NO:25; the VH CDR2 includes the amino acid sequence of SEQ ID NO:75; the VH CDR3 includes the amino acid sequence of SEQ ID NO:27; the VL CDR1 includes the amino acid sequence of SEQ ID NO:76; the VL CDR2 includes the amino acid sequence of SEQ ID NO:29; and the VL CDR3 includes the amino acid sequence of SEQ ID NO:30.

In some embodiments, the VH CDR1 includes the amino acid sequence of SEQ ID NO:25; the VH CDR2 includes the amino acid sequence of SEQ ID NO:26, 31, 34, 35 or 36; the VH CDR3 includes the amino acid sequence of SEQ ID NO:27; the VL CDR1 includes the amino acid sequence of SEQ ID NO:28, 32, 37, 38 or 39; the VL CDR2 includes the amino acid sequence of SEQ ID NO:29; and the VL CDR3 includes the amino acid sequence of SEQ ID NO:30.

In some embodiments, the VH CDR1 comprises the amino acid sequence of SEQ ID NO:13, the VH CDR2 comprises the amino acid sequence of SEQ ID NO:14, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18. In some embodiments, the VH comprises an amino acid sequence selected from the group consisting of SEQ ID NO:1 and 40-43, and the VL comprises an amino acid sequence selected from the group consisting of SEQ ID NO:2 and 44-47. In some embodiments, the VH comprises the amino acid sequence of SEQ ID NO:40, and the VL comprises the amino acid sequence of SEQ ID NO:44.

In some embodiments, the VH CDR1 comprises the amino acid sequence of SEQ ID NO:20, the VH CDR2 comprises the amino acid sequence of SEQ ID NO:21 or 33, the VH CDR3 comprises the amino acid sequence of SEQ ID NO:22, the VL CDR1 comprises the amino acid sequence of SEQ ID NO:23, the VL CDR2 comprises the amino acid sequence of SEQ ID NO:24, and the VL CDR3 comprises the amino acid sequence of SEQ ID NO:18. In some embodiments, the VH CDR1 comprises the amino acid sequence of SEQ ID NO:20, the VH CDR2 comprises the amino acid sequence of SEQ ID NO:33, the VH CDR3 comprises the amino acid sequence of SEQ ID NO:22, the VL CDR1 comprises the amino acid sequence of SEQ ID NO:23, the VL CDR2 comprises the amino acid sequence of SEQ ID NO:24, and the VL CDR3 comprises the amino acid sequence of SEQ ID NO:18. In some embodiments, the VH comprises an amino acid sequence selected from the group consisting of SEQ ID NO:5 and 48-51, and the VL comprises an amino acid sequence selected from the group consisting of SEQ ID NO:6 and 52-55. In some embodiments, the VH comprises the amino acid sequence of SEQ ID NO:48, and the VL comprises the amino acid sequence of SEQ ID NO:52.

In some embodiments, the VH CDR1 comprises the amino acid sequence of SEQ ID NO:25, the VH CDR2 comprises the amino acid sequence of SEQ ID NO:26, 34, 35 or 36, the VH CDR3 comprises the amino acid sequence of SEQ ID NO:27, the VL CDR1 comprises the amino acid sequence of SEQ ID NO:28, 37, 38 or 39, the VL CDR2 comprises the amino acid sequence of SEQ ID NO:29, and the VL CDR3 comprises the amino acid sequence of SEQ ID NO:30. In some embodiments, the VH CDR1 comprises the amino acid sequence of SEQ ID NO:25, the VH CDR2 comprises the amino acid sequence of SEQ ID NO:36, the VH CDR3 comprises the amino acid sequence of SEQ ID NO:27, the VL CDR1 comprises the amino acid sequence of SEQ ID NO:38, the VL CDR2 comprises the amino acid sequence of SEQ ID NO:29, and the VL CDR3 comprises the amino acid sequence of SEQ ID NO:30. In some embodiments, the VH comprises an amino acid sequence selected from the group consisting of SEQ ID NO:9, 56-59 and 64-66, and the VL comprises an amino acid sequence selected from the group consisting of SEQ ID NO:10, 60-63 and 67-69. In some embodiments, the VH comprises the amino acid sequence of SEQ ID NO:66, and the VL comprises the amino acid sequence of SEQ ID NO:68.

In some embodiments, the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18.

In some embodiments, the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:23, the VL CDR2 comprises the amino acid sequence of SEQ ID NO:17, and the VL CDR3 comprises the amino acid sequence of SEQ ID NO:18.

In some embodiments, the VH CDR1 comprises the amino acid sequence of SEQ ID NO:25, the VH CDR2 comprises the amino acid sequence of SEQ ID NO:31, the VH CDR3 comprises the amino acid sequence of SEQ ID NO:27, the VL CDR1 comprises the amino acid sequence of SEQ ID NO:32, the VL CDR2 comprises the amino acid sequence of SEQ ID NO:29, and the VL CDR3 comprises the amino acid sequence of SEQ ID NO:30.

Treatment methods and uses are also provided. In one embodiment, a method of treating cancer in a patient in need thereof is provided, comprising administering to the patient an effective amount of the antibody or the polypeptide of the present disclosure. In some embodiments, the cancer is a solid tumor. In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer, in particular ovarian cancer and colorectal cancer.

### BRIEF DESCRIPTION OF THE DRAWINGS

FIG. 1A shows the binding affinity of each antibody to human AMHR2 expressed on CHO-K1 cells.

FIG. 1B shows the binding affinity of each antibody to human AMHR2 expressed on Rko cells.

FIG. 2 shows the ADCC of each antibody to AMHR2-expressing CHO-K1 cells.

FIG. 3 shows the endocytosis of each antibody by AMHR2-expressing CHO-K1 cells.

FIG. 4 shows the cytotoxic activity of each antibody on AMHR2-expressing Rko cells.

FIG. 5A-H show the binding affinity of each humanized antibody to human AMHR2 expressed on CHO-K1 cells.

FIG. 6 shows FACS binding of some of the humanized antibodies to AMHR2.

FIG. 7A-B show the binding affinity of the selected humanized antibodies to human AMHR2 expressed on CHO-K1 and Rko cells.

FIG. 8A-B show the ADCC efficacy of the selected humanized antibodies to human AMHR2-expressing CHO-K1 and Rko cells.

FIG. 9A-B show the endocytosis inducing activity of the selected humanized antibodies on human AMHR2-expressing CHO-K1 and Rko cells.

FIG. 10A-C show the cross-reactivity of the selected humanized antibodies could to the cyno, rat, and mouse AMHRII proteins.

FIG. 11A-B show the killing activity of AMHR2-expressing CHO-K1 and Rko cells by the selected humanized antibodies.

### DETAILED DESCRIPTION

### Definitions

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Anti-AMHRII Antibodies

The present disclosure provides antibodies and antigen-binding fragments capable of binding the human AMHRII protein, which binding can effectively induce endocytosis by the targeted cell that expresses the AMHRII protein. Also, these antibodies demonstrate potent cytotoxic activities, making them suitable for killing the target cell. Such target cells include cancer cells, such as epithelial ovarian carcinoma (EOC) cells that overexpress the AMHRII protein.

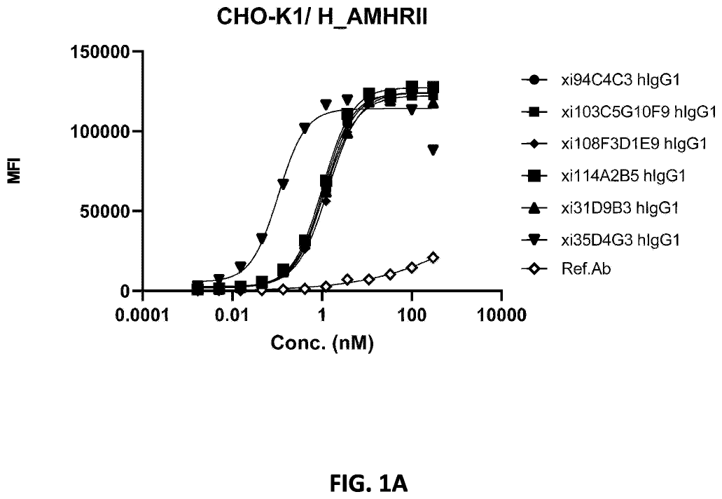

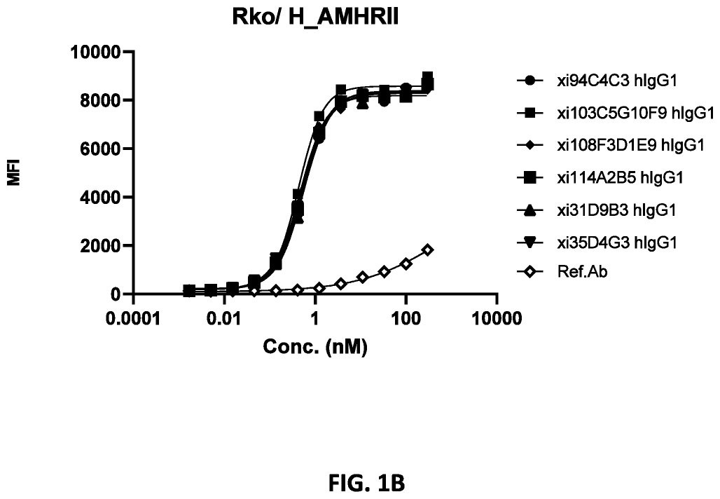

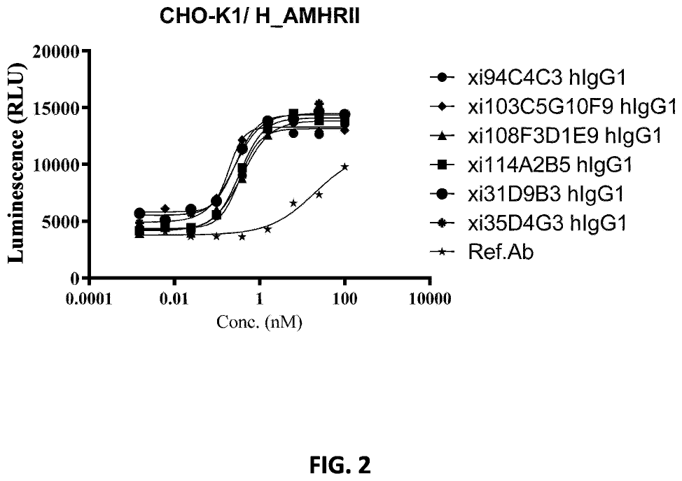

More specifically, all of the six murine antibodies (in chimeric forms) prepared herein exhibited considerably higher binding affinity to AMHRII expressed on CHO and Ryo cells, as compared to the positive control Ref.Ab (FIG. 1A-B). Likely due to such strong bindings, these antibodies also exhibited significantly higher ADCC, endocytosis and cytotoxic activities than the reference (FIG. 2-4).

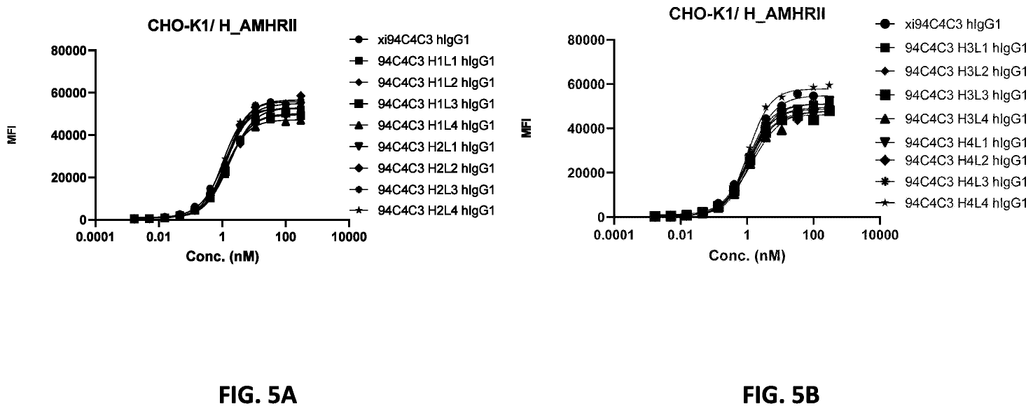

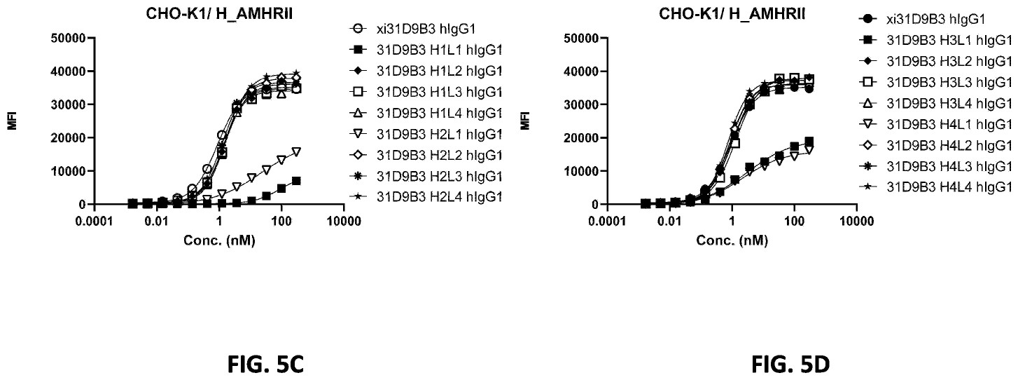

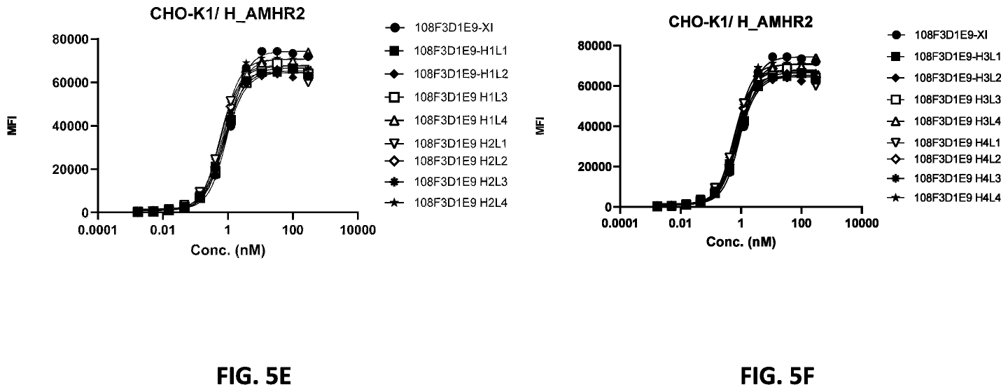

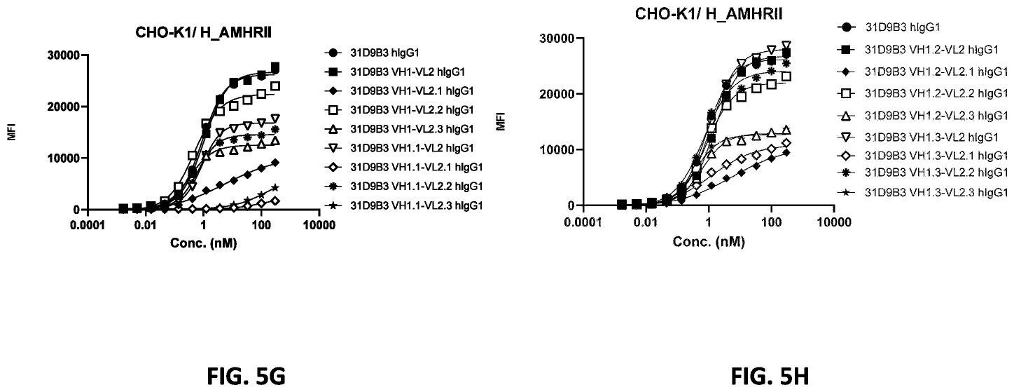

Moreover, these excellent properties were retained through the humanization and post-translational modification (PTM) de-risking processes. All of the humanized versions of 94C4C3 and 108F3D1E9, and most of 31D9B3 retained the high binding affinities and endocytosis activities (see, e.g., FIGS. 5A-H, and 9A-B).

In accordance with one embodiment of the present disclosure, therefore, provided is an antibody or antigen-binding fragment selected from those provided in Table 1, as well as their derivatives and humanized ones, such as those that include the CDR regions (see, e.g., Table 1A) of these antibodies, as well as those that include variants of these CDR regions, such as PTM de-risked ones.

It is observed that four of the antibodies in Table 1, including 94C4C3, 103C5G10F9, 108F3D1E9, and 114A2B5, have high sequence homology among them. Their CDR sequences also share high sequence identity. Accordingly, it is contemplated that these CDR sequences are interchangeable while retaining good binding affinity.

Accordingly, one embodiment of the present disclosure provides an antibody or antigen-binding fragment thereof that includes a heavy chain variable region (VH) comprising a VH CDR1, VH CDR2, and VH CDR3, and a light chain variable region (VL) comprising a VL CDR1, VL CDR2 and VL CDR3, wherein:
the VH CDR1 includes the amino acid sequence of SEQ ID NO:13 or 20;
the VH CDR2 includes the amino acid sequence of SEQ ID NO:14, 19, 21 or 33;
the VH CDR3 includes the amino acid sequence of SEQ ID NO:15 or 22;
the VL CDR1 includes the amino acid sequence of SEQ ID NO:16 or 23;
the VL CDR2 includes the amino acid sequence of SEQ ID NO:17 or 24; and
the VL CDR3 includes the amino acid sequence of SEQ ID NO:18.

The sequences can also be represented with degenerated ones, such as those illustrated in Table A. Accordingly, one embodiment of the present disclosure provides an antibody or antigen-binding fragment thereof that includes a heavy chain variable region (VH) comprising a VH CDR1, VH CDR2, and VH CDR3, and a light chain variable region (VL) comprising a VL CDR1, VL CDR2 and VL CDR3, wherein:
the VH CDR1 includes the amino acid sequence of SEQ ID NO:70;
the VH CDR2 includes the amino acid sequence of SEQ ID NO:71;
the VH CDR3 includes the amino acid sequence of SEQ ID NO:72;
the VL CDR1 includes the amino acid sequence of SEQ ID NO:73;
the VL CDR2 includes the amino acid sequence of SEQ ID NO:74; and
the VL CDR3 includes the amino acid sequence of SEQ ID NO: 18.

TABLE A
CDR Sequences of the Antibody Group I

| | | SEQ ID |
|---|---|---|
| CDR | Sequence | NO: |
| VH CDR1 | SDYAWN | 13 |
| | SDFAWN | 20 |
| | SDXAWN (X is F or Y) | 70 |
| VH CDR2 | YISYSGSISYNPSLKS | 14 |
| | YISYSGSTSYNPSLKS | 19 |
| | YINFSGSTSYNPSLKS | 21 |
| | YISFSGSTSYNPSLKS | 33 |
| | YI [symbol] SGS [symbol] SYNPSLKS | 71 |
| | (X₁ is N or S, X₂ is F | |
| | or Y, X₃ is I or T) | |
| VH CDR3 | SRYSWFDY | 15 |
| | SRYSWFAY | 22 |
| | SRYSWFXY (X is A or D) | 72 |
| VL CDR1 | SASESVDTYGNSFMH | 16 |
| | RASESVDTYGNSFMH | 23 |
| | XASESVDTYGNSFMH (X is R or S) | 73 |
| VL CDR2 | RASSLES | 17 |
| | RASNLES | 24 |
| | RASXLES (X is N or S) | 74 |
| VL CDR3 | QQSNEDPYT | 18 |

Likewise, antibodies 31D9B3 and 35D4G3 also share high sequence identity between the VH and VL sequences and their CDR regions. Accordingly, one embodiment of the present disclosure provides an antibody or antigen-binding fragment thereof that includes a heavy chain variable region (VH) comprising a VH CDR1, VH CDR2, and VH CDR3, and a light chain variable region (VL) comprising a VL CDR1, VL CDR2 and VL CDR3, wherein:
the VH CDR1 includes the amino acid sequence of SEQ ID NO:25;
the VH CDR2 includes the amino acid sequence of SEQ ID NO:26, 31, 34, 35 or 36;
the VH CDR3 includes the amino acid sequence of SEQ ID NO:27;
the VL CDR1 includes the amino acid sequence of SEQ ID NO:28, 32, 37, 38 or 39;
the VL CDR2 includes the amino acid sequence of SEQ ID NO:29; and
the VL CDR3 includes the amino acid sequence of SEQ ID NO:30.

The sequences can also be represented with degenerated ones, such as those illustrated in Table B. Accordingly, one embodiment of the present disclosure provides an antibody or antigen-binding fragment thereof that includes a heavy chain variable region (VH) comprising a VH CDR1, VH CDR2, and VH CDR3, and a light chain variable region (VL) comprising a VL CDR1, VL CDR2 and VL CDR3, wherein:
the VH CDR1 includes the amino acid sequence of SEQ ID NO:25;
the VH CDR2 includes the amino acid sequence of SEQ ID NO:75;
the VH CDR3 includes the amino acid sequence of SEQ ID NO:27;
the VL CDR1 includes the amino acid sequence of SEQ ID NO:76;
the VL CDR2 includes the amino acid sequence of SEQ ID NO:29; and
the VL CDR3 includes the amino acid sequence of SEQ ID NO:30.

TABLE B
CDR Sequences of the Antibody Group II

| | | SEQ ID |
|---|---|---|
| CDR | Sequence | NO: |
| VH CDR1 | DYWVS | 25 |
| VH CDR2 | EIYPKNGVSNFNEKFKG | 26 |
| | EIYPDNGITNFNEKFKG | 31 |
| | EIYPKEGVSNFNEKFKG | 34 |
| | EIYPKNDVSNFNEKFKG | 35 |
| | EIYPKNQVSNFNEKFKG | 36 |
| | EIYP [symbol] NFNEKFKG | 75 |
| | (X₁ is D or K, X₂ is | |
| | E or N, X₃ is D, G or Q, | |
| | X₄ is I or V, X₅ is S or T) | |
| VH CDR3 | QRDY | 27 |
| VL CDR1 | RSSQSLEYGDGYTYLE | 28 |
| | RSSQSLKYGDGYTYLE | 32 |
| | RSSQSLEYGSGYTYLE | 37 |
| | RSSQSLEYGDRYTYLE | 38 |
| | RSSQSLEYGTGYTYLE | 39 |
| | RSSQSL [symbol] YG [symbol] YTYLE | 76 |
| | (X₁ is E or K, X₂ is D, S | |
| | or T, X₃ is G or R) | |
| VL CDR2 | GVSNRFS | 29 |
| VL CDR3 | FQATHDPNT | 30 |

In one embodiment, the present disclosure provides an antibody or antigen-binding fragment thereof that includes the CDRs of 94C4C3, which has a heavy chain variable region (VH) having the sequence of SEQ ID NO:1 and a light chain variable region (VL) having the sequence of SEQ ID NO:2.

In some embodiments, the VH CDR1 includes the amino acid sequence of SEQ ID NO:13, the VH CDR2 includes the amino acid sequence of SEQ ID NO:14, the VH CDR3 includes the amino acid sequence of SEQ ID NO:15, the VL CDR1 includes the amino acid sequence of SEQ ID NO:16, the VL CDR2 includes the amino acid sequence of SEQ ID NO:17, and the VL CDR3 includes the amino acid sequence of SEQ ID NO:18.

Example antibodies and fragments having these CDRs include those having a VH having an amino acid sequence selected from the group consisting of SEQ ID NO:1 and 40-43 (or an amino acid sequence having at least 80%, 85%, 90%, 95%, 98% or 99% sequence identity to any of them while keeping the respective CDR sequences), and a VL having an amino acid sequence selected from the group consisting of SEQ ID NO:2 and 44-47 (or an amino acid sequence having at least 80%, 85%, 90%, 95%, 98% or 99% sequence identity to any of them while keeping the respective CDR sequences). Example humanized antibodies and fragments having these CDRs include those having a VH having an amino acid sequence selected from the group consisting of SEQ ID NO:40-43, and a VL having an amino acid sequence selected from the group consisting of SEQ ID NO:44-47. In a particular embodiment, the VH includes the amino acid sequence of SEQ ID NO:40, and the VL includes the amino acid sequence of SEQ ID NO:44.

In one embodiment, the present disclosure provides an antibody or antigen-binding fragment thereof that includes the CDRs of 108F3D1E9, which has a heavy chain variable region (VH) having the sequence of SEQ ID NO:5 and a light chain variable region (VL) having the sequence of SEQ ID NO:6.

In some embodiments, the VH CDR1 includes the amino acid sequence of SEQ ID NO:20, the VH CDR2 includes the amino acid sequence of SEQ ID NO:21, the VH CDR3 includes the amino acid sequence of SEQ ID NO:22, the VL CDR1 includes the amino acid sequence of SEQ ID NO:23, the VL CDR2 includes the amino acid sequence of SEQ ID NO:24, and the VL CDR3 includes the amino acid sequence of SEQ ID NO:18.

In some embodiments, the VH CDR1 includes the amino acid sequence of SEQ ID NO:20, the VH CDR2 is PTM de-risked and includes the amino acid sequence of SEQ ID NO:33, the VH CDR3 includes the amino acid sequence of SEQ ID NO:22, the VL CDR1 includes the amino acid sequence of SEQ ID NO:23, the VL CDR2 includes the amino acid sequence of SEQ ID NO:24, and the VL CDR3 includes the amino acid sequence of SEQ ID NO:18.

Example antibodies and fragments having these CDRs include those having a VH having an amino acid sequence selected from the group consisting of SEQ ID NO:5 and 48-51 (or an amino acid sequence having at least 80%, 85%, 90%, 95%, 98% or 99% sequence identity to any of them while keeping the respective CDR sequences), and a VL having an amino acid sequence selected from the group consisting of SEQ ID NO:6 and 52-55 (or an amino acid sequence having at least 80%, 85%, 90%, 95%, 98% or 99% sequence identity to any of them while keeping the respective CDR sequences). Example humanized and PTM de-risked antibodies and fragments having these CDRs include those having a VH having an amino acid sequence selected from the group consisting of SEQ ID NO: 48-51, and a VL having an amino acid sequence selected from the group consisting of SEQ ID NO:52-55. In some embodiments, the VH includes the amino acid sequence of SEQ ID NO:48, and the VL includes the amino acid sequence of SEQ ID NO:52.

In one embodiment, the present disclosure provides an antibody or antigen-binding fragment thereof that includes the CDRs of 31D9B3, which has a heavy chain variable region (VH) having the sequence of SEQ ID NO:9 and a light chain variable region (VL) having the sequence of SEQ ID NO:10.

In some embodiments, the VH CDR1 includes the amino acid sequence of SEQ ID NO:25, the VH CDR2 includes the amino acid sequence of SEQ ID NO:26, the VH CDR3 includes the amino acid sequence of SEQ ID NO:27, the VL CDR1 includes the amino acid sequence of SEQ ID NO:28, the VL CDR2 includes the amino acid sequence of SEQ ID NO:29, and the VL CDR3 includes the amino acid sequence of SEQ ID NO:30.

In some embodiments, the VH CDR1 includes the amino acid sequence of SEQ ID NO:25, the VH CDR2 includes the amino acid sequence of SEQ ID NO:26, or a PTM de-risked version of SEQ ID NO:34, 35 or 36, the VH CDR3 includes the amino acid sequence of SEQ ID NO:27, the VL CDR1 includes the amino acid sequence of SEQ ID NO:28, or a PTM de-risked version of SEQ ID NO:37, 38 or 39, the VL CDR2 includes the amino acid sequence of SEQ ID NO:29, and the VL CDR3 includes the amino acid sequence of SEQ ID NO:30.

In some embodiments, the VH CDR1 includes the amino acid sequence of SEQ ID NO:25, the VH CDR2 includes the amino acid sequence of SEQ ID NO:34, 35 or 36, the VH CDR3 includes the amino acid sequence of SEQ ID NO:27, the VL CDR1 includes the amino acid sequence of SEQ ID NO:37, 38 or 39, the VL CDR2 includes the amino acid sequence of SEQ ID NO:29, and the VL CDR3 includes the amino acid sequence of SEQ ID NO:30.

In some embodiments, the VH CDR1 includes the amino acid sequence of SEQ ID NO:25, the VH CDR2 includes the amino acid sequence of SEQ ID NO:36, the VH CDR3 includes the amino acid sequence of SEQ ID NO:27, the VL CDR1 includes the amino acid sequence of SEQ ID NO:38, the VL CDR2 includes the amino acid sequence of SEQ ID NO:29, and the VL CDR3 includes the amino acid sequence of SEQ ID NO:30.

Example antibodies and fragments having these CDRs include those having a VH having an amino acid sequence selected from the group consisting of SEQ ID NO:9, 56-59 and 64-66 (or an amino acid sequence having at least 80%, 85%, 90%, 95%, 98% or 99% sequence identity to any of them while keeping the respective CDR sequences), and a VL having an amino acid sequence selected from the group consisting of SEQ ID NO:10, 60-63 and 67-69 (or an amino acid sequence having at least 80%, 85%, 90%, 95%, 98% or 99% sequence identity to any of them while keeping the respective CDR sequences). Example humanized antibodies and fragments having these CDRs include those having a VH having an amino acid sequence selected from the group consisting of SEQ ID NO:56-59 and 64-66, and a VL having an amino acid sequence selected from the group consisting of SEQ ID NO:60-63 and 67-69. Example humanized and PTM de-risked antibodies and fragments having these CDRs include those having a VH having an amino acid sequence selected from the group consisting of SEQ ID NO:64-66, and a VL having an amino acid sequence selected from the group consisting of SEQ ID NO:67-69. In some embodiments, the VH includes the amino acid sequence of SEQ ID NO:66, and the VL includes the amino acid sequence of SEQ ID NO:68.

In one embodiment, the present disclosure provides an antibody or antigen-binding fragment thereof that includes the CDRs of 103C5G10F9, which has a heavy chain variable region (VH) having the sequence of SEQ ID NO:3 and a light chain variable region (VL) having the sequence of SEQ ID NO:4.

In some embodiments, the VH CDR1 includes the amino acid sequence of SEQ ID NO:13, the VH CDR2 includes the amino acid sequence of SEQ ID NO:19, the VH CDR3 includes the amino acid sequence of SEQ ID NO:15, the VL CDR1 includes the amino acid sequence of SEQ ID NO:16, the VL CDR2 includes the amino acid sequence of SEQ ID NO:17, and the VL CDR3 includes the amino acid sequence of SEQ ID NO:18.

In one embodiment, the present disclosure provides an antibody or antigen-binding fragment thereof that includes the CDRs of 114A2B5, which has a heavy chain variable region (VH) having the sequence of SEQ ID NO:7 and a light chain variable region (VL) having the sequence of SEQ ID NO:8.

In some embodiments, the VH CDR1 includes the amino acid sequence of SEQ ID NO:13, the VH CDR2 includes the amino acid sequence of SEQ ID NO:19, the VH CDR3 includes the amino acid sequence of SEQ ID NO:15, the VL CDR1 includes the amino acid sequence of SEQ ID NO:23, the VL CDR2 includes the amino acid sequence of SEQ ID NO:17, and the VL CDR3 includes the amino acid sequence of SEQ ID NO:18.

In one embodiment, the present disclosure provides an antibody or antigen-binding fragment thereof that includes the CDRs of 35D4G3, which has a heavy chain variable region (VH) having the sequence of SEQ ID NO:11 and a light chain variable region (VL) having the sequence of SEQ ID NO:12.

In some embodiments, the VH CDR1 includes the amino acid sequence of SEQ ID NO:25, the VH CDR2 includes the amino acid sequence of SEQ ID NO:31, the VH CDR3 includes the amino acid sequence of SEQ ID NO:27, the VL CDR1 includes the amino acid sequence of SEQ ID NO:32, the VL CDR2 includes the amino acid sequence of SEQ ID NO:29, and the VL CDR3 includes the amino acid sequence of SEQ ID NO:30.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

### Bispecific and Multispecific Antibodies, and Chimeric Antigen Receptors (CAR)

As provided, the anti-AMHRII antibodies or fragments disclosed here can also be useful for preparing bispecific and multispecific antibodies, as well as chimeric antigen receptors (CAR).

Accordingly, in one embodiment, provided is a bispecific antibody that includes an anti-AMHRII antibody of the present disclosure, or an antigen-binding fragment thereof, and a second antibody or antigen-binding fragment having binding specificity to a target antigen that is not AMHRII. In some embodiment, a third or fourth specificity is further included.

The target antigen that is not AMHRII, in some embodiments, is a tumor antigen. An abundance of tumor antigens are known in the art and new tumor antigens can be readily identified by screening. Non-limiting examples of tumor antigens include ABL, ALK, B4GALNT1, BAFF, BCL2, BRAF, BTK, CD19, CD20, CD30, CD38, CD52, CD73, Claudin 18.2, CTLA-4, EGFR, FOLR1, FLT3, HDAC, HER2, IDH2, IL-1β, IL-6, IL-6R, JAK1/2, JAK3, KIT, LAG-3, MEK, Nectin 4, ROR1, mTOR, PARP, PD-1, PDGFR, PDGFRα, PD-L1, PI3K6, PIGF, PTCH, RAF, RANKL, Smoothened, VEGF, VEGFR, and VEGFR2. Other examples are Her2, EpCAM, CD33, CD47, CD133, CEA, gpA33, Mucins, TAG-72, CIX, PSMA, GD2, GD3, GM2, Integrin, αVβ, α5β1, ERBB2, ERBB3, MET, IGF1R, EPHA3, TRAILR1, TRAILR2, RANKL, FAP and Tenascin.

Also provided, are chimeric antigen receptor (CAR) that includes an antibody or fragment of the present disclosure. In the CAR, the antibody or fragment can serve as the antigen recognition domain. In addition, in some embodiments, the CAR also includes an extracellular hinge region, a transmembrane domain, and an intracellular T-cell signaling domain.

The hinge, also called a spacer, is a small structural domain that sits between the antigen recognition region and the cell's outer membrane. A suitable hinge enhances the flexibility of the scFv receptor head, reducing the spatial constraints between the CAR and its target antigen. Example hinge sequences are based on membrane-proximal regions from immune molecules such as IgG, CD8, and CD28.

The transmembrane domain is a structural component, consisting of a hydrophobic alpha helix that spans the cell membrane. It anchors the CAR to the plasma membrane, bridging the extracellular hinge and antigen recognition domains with the intracellular signaling region. Typically, the transmembrane domain from a membrane-proximal component of the endodomain can be used, such as the CD28 transmembrane domain.

The intracellular T-cell signaling domain lies in the receptor's endodomain, inside the cell. After an antigen is bound to the external antigen recognition domain, CAR receptors cluster together and transmit an activation signal. Then the internal cytoplasmic end of the receptor perpetuates signaling inside the T cell. To mimic this process, CD3-zeta's cytoplasmic domain is commonly used as the main CAR endodomain component.

T cells also require co-stimulatory molecules in addition to CD3 signaling in order to persist after activation. In some embodiments, the endodomains of CAR receptor also includes one or more chimeric domains from co-stimulatory proteins, such as CD28, CD27, CD134 (OX40), and CD137 (4-1BB).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Cancer Treatment

As described herein, the antibodies, bispecific antibodies, polypeptides, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

In some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient over-express AMHRII.

Also provided are methods for killing a cell that expresses AMHRII. The method, in one embodiment, entails contacting the cell with an antibody or antigen-binding fragment of the present disclosure. The contacting may be in vivo, ex vivo or in vitro. Such a cell may be a cancer cell or a cell that otherwise naturally is engineered to express AMHRII.

Cellular therapies, such as chimeric antigen receptor (CAR) T-cell or NK cell therapies, are also provided in the present disclosure. A suitable cell can be used, that is put in contact with an antibody or CAR of the present disclosure (or alternatively engineered to express an antibody or CAR of the present disclosure). Upon such contact or engineering, the cell can then be introduced to a cancer patient in need of a treatment. The cancer patient may have a cancer of any of the types as disclosed herein. The cell (e.g., T cell or NK cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

### Diagnostic Methods

Over-expression of AMHRII is observed in certain tumor samples, and patients having AMHRII-over-expressing cells are likely responsive to treatments with the anti-AMHRII antibodies of the present disclosure. Accordingly, the antibodies of the present disclosure can also be used for diagnostic and prognostic purposes.

A sample that preferably includes a cell can be obtained from a patient, which can be a cancer patient or a patient desiring diagnosis. The cell be a cell of a tumor tissue or a tumor block, a blood sample, a urine sample or any sample from the patient. Upon optional pre-treatment of the sample, the sample can be incubated with an antibody of the present disclosure under conditions allowing the antibody to interact with a AMHRII protein potentially present in the sample. Methods such as ELISA can be used, taking advantage of the anti-AMHRII antibody, to detect the presence of the AMHRII protein in the sample.

Presence of the AMHRII protein in the sample (optionally with the amount or concentration) can be used for diagnosis of cancer, as an indication that the patient is suitable for a treatment with the antibody, or as an indication that the patient has (or has not) responded to a cancer treatment. For a prognostic method, the detection can be done at once, twice or more, at certain stages, upon initiation of a cancer treatment to indicate the progress of the treatment.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1: Generation of Murine Antibodies Against Human AMHRII

The human AMHRII protein was used to immunize different strains of mice, and hybridomas were generated accordingly. AMHRII positive binders were selected and subcloned. Subsequently, in vitro binding and functional screening were carried out and lead antibodies with highest binding affinity and strongest functional potency were identified.

The VH/VL sequences of the lead murine antibodies are provided in Table 1 below.

TABLE 1
Sequences of the selected clones

| | | SEQ ID |
|---|---|---|
| Antibody | Sequence | NO: |
| 94C4C3 VH | DVQLQESGPGLVKPSQSLSLTCTVTGYSITSDYAWNWIRQFPGNKLEWMG | 1 |
| | YISYSGSISYNPSLKSRISITRDTSKNQFFLQLNSVTTEDTATYYCARSR | |
| | YSWFDYWGQGTLVTVSA | |
| 94C4C3 VL | DIVLTQSPTSLAVSLGQRATLSCSASESVDTYGNSFMHWYQQKPGQPPKL | 2 |
| | LIYRASSLESGIPVRFSGSGSRTDFTLTINPVEAADVATYYCQQSNEDPY | |
| | TFGGGTKLEIK | |
| 103C5G10F9 VH | DVQLQESGPGLVKPSQSLSLTCTVTGYSITSDYAWNWIRQFPGNKLEWMG | 3 |
| | YISYSGSTSYNPSLKSRISITRDTSKNQFFLQLNSVTTEDTATYYCARSR | |
| | YSWFDYWGQGTLVTVSA | |
| 103C5G10F9 VL | DIVLTQSPTSLAVSLGQRATLSCSASESVDTYGNSFMHWYQQKPGQSPKL | 4 |
| | LIYRASSLESGIPARFSGSGSRTDFTLTINPVEAADVATYYCQQSNEDPY | |
| | TFGGGTKLEIK | |
| 108F3D1E9 VH | DVQLQESGPGLVKPSQSLSLTCTVTGYSITSDFAWNWIRQFPGNKLEWMG | 5 |
| | YINFSGSTSYNPSLKSRISITRDTSKNQFFLQLNSVTTEDTATYYCAGSR | |
| | YSWFAYWGQGTLVTVSA | |
| 108F3D1E9 VL | DIVLTQSPASLAVSLGQRATISCRASESVDTYGNSFMHWYQQKPGQPPKL | 6 |
| | LIYRASNLESGIPARFSGSGSRTDFTLTINPVEADDVATYYCQQSNEDPY | |
| | TFGGGTKLEIK | |
| 114A2B5 VH | DVQLQESGPGLVKPSQSLSLTCTVTGYSITSDYAWNWIRQFPGNKLEWMG | 7 |
| | YISYSGSTSYNPSLKSRISITRDTSKNQFFLQLNSVTTEDTATYYCARSR | |
| | YSWFDYWGQGTLVTVSA | |
| 114A2B5 VL | DIVLTQSPASLAVSLGQRATLSCRASESVDTYGNSFMHWYQQKPGQSPKL | 8 |
| | LIYRASSLESGIPARFSGSGSRTDFTLTINPVEAADVATYYCQQSNEDPY | |
| | TFGGGTKLEIK | |
| 31D9B3 VH | QVKLLQSGAALVKPGASVKMSCKASGYTFTDYWVSWVKQSHGKSLEWIGE | 9 |
| | IYPKNGVSNFNEKFKGKATLTVDKSTSTAYMELRRLTSEDSAIFYCTRQR | |
| | DYWGQGVMVTVSS | |
| 31D9B3 VL | DVVLTQTPGSLSVTLGDQASISCRSSQSLEYGDGYTYLEWYLQKPGQSPQ | 10 |
| | LLIYGVSNRFSGVPDRFIGSGSGTDFTLKISRVEPEDLGVYYCFQATHDP | |
| | NTFGAGTKLELK | |
| 35D4G3 VH | QVRLLQSGAALVKPGASAKMSCKASGYTFTDYWVSWVKQSHGKSLEWIGE | 11 |
| | IYPDNGITNFNEKFKGKATLTVDKSTRTAYMELSRLTSEDSAIYYCTRQR | |
| | DYWGQGVMVTVSS | |
| 35D4G3 VL | DVVLTQTPGSLSVTLGDQVSISCRSSQSLKYGDGYTYLEWYLQKPGQSPQ | 12 |
| | LLFYGVSNRFSGVPDRFIGSGSGTDFTLKISRVEPEDLGVYYCFQATHDP | |
| | NTFGTGTKLDLK | |

Their CDR sequences are summarized in Table 1A below.

TABLE 1A
Sequences of the CDRs

| | | | SEQ |
|---|---|---|---|
| | | | ID |
| Antibody | CDR | Sequence | NO: |
| 94C4C3 | VH CDR1 | SDYAWN | 13 |
| | VH CDR2 | YISYSGSISYNPSLKS | 14 |
| | VH CDR3 | SRYSWFDY | 15 |
| | VL CDR1 | SASESVDTYGNSFMH | 16 |
| | VL CDR2 | RASSLES | 17 |
| | VL CDR3 | QQSNEDPYT | 18 |
| 103C5G10F9 | VH CDR1 | SDYAWN | 13 |
| | VH CDR2 | YISYSGSTSYNPSLKS | 19 |
| | VH CDR3 | SRYSWFDY | 15 |
| | VL CDR1 | SASESVDTYGNSFMH | 16 |
| | VL CDR2 | RASSLES | 17 |
| | VL CDR3 | QQSNEDPYT | 18 |
| 108F3D1E9 | VH CDR1 | SDFAWN | 20 |
| | VH CDR2 | YINFSGSTSYNPSLKS | 21 |
| | VH CDR3 | SRYSWFAY | 22 |
| | VL CDR1 | RASESVDTYGNSFMH | 23 |
| | VL CDR2 | RASNLES | 24 |
| | VL CDR3 | QQSNEDPYT | 18 |
| 114A2B5 | VH CDR1 | SDYAWN | 13 |
| | VH CDR2 | YISYSGSTSYNPSLKS | 19 |
| | VH CDR3 | SRYSWFDY | 15 |
| | VL CDR1 | RASESVDTYGNSFMH | 23 |
| | VL CDR2 | RASSLES | 17 |
| | VL CDR3 | QQSNEDPYT | 18 |
| 31D9B3 | VH CDR1 | DYWVS | 25 |
| | VH CDR2 | EIYPKNGVSNFNEKFKG | 26 |
| | VH CDR3 | QRDY | 27 |
| | VL CDR1 | RSSQSLEYGDGYTYLE | 28 |
| | VL CDR2 | GVSNRFS | 29 |
| | VL CDR3 | FQATHDPNT | 30 |
| 35D4G3 | VH CDR1 | DYWVS | 25 |
| | VH CDR2 | EIYPDNGITNFNEKFKG | 31 |
| | VH CDR3 | QRDY | 27 |
| | VL CDR1 | RSSQSLKYGDGYTYLE | 32 |
| | VL CDR2 | GVSNRFS | 29 |
| | VL CDR3 | FQATHDPNT | 30 |

Antibodies 94C4C3, 103C5G10F9, 108F3D1E9 and 114A2B5 appear to share similar CDRs, while the 31D9B3 and 35D4G3 also have homologous CDRs.

### Example 2. Binding of Chimeric Antibodies on AMHRII-Overexpressed Cells

This example tested the binding affinities of the selected antibodies, in human chimeric format, in binding to the human AMHRII protein expressed on cells.

CHO-K1 cells that over-expressed AMHR2 were harvested from flasks. 100 μl of 1×10⁶ cells/ml of cells were incubated with primary chimeric antibodies (or with positive reference antibody Ref.Ab) in 3-fold serial dilutions starting from 300 nM to 0.002 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

As shown in FIG. 1A and Table 2, the results showed that the chimeric antibodies can bind to human AMHR2 with lower EC50 (higher affinity) than the reference antibody Ref.Ab.

TABLE 2
Binding Affinity to CHO Cell-expressed AMHRII

| Antibody | EC₅₀ (nM) | Top (MFI) |
|---|---|---|
| xi94C4C3 hIgG1 | 1.17 | 124288 |
| xi103C5G10F9 hIgG1 | 1.08 | 123877 |
| xi108F3D1E9 hIgG1 | 1.37 | 124287 |
| xi114A2B5 hIgG1 | 1.04 | 127454 |
| xi31D9B3 hIgG1 | 1.16 | 122404 |
| xi35D4G3 hIgG1 | 0.11 | 114173 |
| Ref.Ab | NA | 91173 |

Similarly, the binding of these antibodies was tested with Rko cells that over-expressed AMHR2. 100 μl of 1×10⁶ cells/ml of cells were incubated with the antibodies in 3-fold serial dilutions starting from 300 nM to 0.002 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

As shown in FIG. 1B and Table 3, the results showed that the chimeric antibodies can bind to human AMHR2 with lower EC50 (higher affinity) than the reference antibody Ref.Ab.

TABLE 3
Binding Affinity to Rko Cell-expressed AMHRII

| Antibody | EC₅₀ (nM) | Top (MFI) |
|---|---|---|
| xi94C4C3 hIgG1 | 0.56 | 8384 |
| xi103C5G10F9 hIgG1 | 0.43 | 8578 |
| xi108F3D1E9 hIgG1 | 0.47 | 8187 |
| xi114A2B5 hIgG1 | 0.52 | 8327 |
| xi31D9B3 hIgG1 | 0.57 | 8360 |
| xi35D4G3 hIgG1 | 0.49 | 8294 |
| Ref.Ab | 575.5 | 4142 |

### Example 3. ADCC of Chimeric Antibodies on AMHRII-Overexpressed Cells

This example tested the antibody-dependent cellular cytotoxicity (ADCC) of the chimeric antibodies.

The ADCC Reporter Bioassay used here uses an alternative readout at an earlier point in ADCC MOA pathway activation: the activation of gene transcription through the NFAT (nuclear factor of activated T-cells) pathway in the effector cell. In addition, the ADCC Reporter Bioassay uses engineered Jurkat cells stably expressing the FcγRIIIa receptor, V158 (high affinity) variant, and an NFAT response element driving expression of firefly luciferase as effector cells. Antibody biological activity in ADCC MOA is quantified through the luciferase produced as a result of NFAT pathway activation; luciferase activity in the effector cell is quantified with luminescence readout.

Serial dilutions of AMHR2 chimeric monoclonal antibody were incubated for 6 hours of induction at 37° C. with engineered Jurkat effector cells (ADCC Bioassay Effector Cells), with ADCC Bioassay Target Cells (expressing AMHR2). Luciferase activity was quantified using Bio-Glo™ Reagent.

The results are presented in FIG. 2 and Table 4, which show that these chimeric antibodies have stronger ADCC activities than the reference antibody Ref.Ab.

TABLE 4
ADCC Efficacy of the Antibodies

| Antibody | EC₅₀ (nM) | Top (MFI) |
|---|---|---|
| xi94C4C3 hIgG1 | 0.35 | 13142 |
| xi103C5G10F9 hIgG1 | 0.39 | 13854 |
| xi108F3D1E9 hIgG1 | 0.19 | 13288 |
| xi114A2B5 hIgG1 | 0.34 | 14100 |
| xi31D9B3 hIgG1 | 0.27 | 14367 |
| xi35D4G3 hIgG1 | 0.25 | 14487 |
| Ref.Ab | 19.82 | 11202 |

### Example 4. Internalization of Chimeric Antibody by AMHRII-Overexpressed Cells

This example tested the chimeric antibodies' ability to induce endocytosis by cells overexpressing AMHRII.

The method used here employs a new hydrophilic and bright pH sensor dye (pHAb dye), which is not fluorescent at neutral pH but becomes highly fluorescent at acidic pH with internalization. It can be used to detect the process of endocytosis. CHO-K1 cells over-expressed human AMHR2 as the target cells, the detection antibody labeled with pHAb dye was added to evaluate the endocytosis of AMHR2 chimeric monoclonal antibody in vitro.

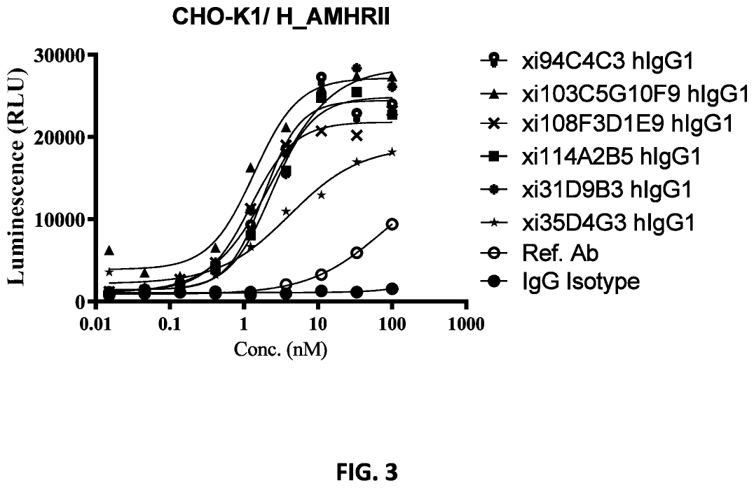

Serial dilutions of AMHR2 chimeric monoclonal antibody were incubated for 24 hours at 37° C. Luciferase activity was detected. The results are presented in FIG. 3 and Table 5, which show that these chimeric antibodies have stronger endocytosis-inducing activities, as compared to Ref.Ab.

TABLE 5
Endocytosis of the Antibodies

| Antibody | EC₅₀ (nM) | Top (MFI) |
|---|---|---|
| xi94C4C3 hIgG1 | 1.86 | 24426 |
| xi103C5G10F9 hIgG1 | 1.37 | 27164 |
| xi108F3D1E9 hIgG1 | 1.23 | 21809 |
| xi114A2B5 hIgG1 | 2.34 | 24848 |
| xi31D9B3 hIgG1 | 2.47 | 28401 |
| xi35D4G3 hIgG1 | 4.14 | 18869 |
| Ref. Ab | 73.28 | 15896 |
| IgG Isotype | NA | NA |

### Example 5. Cytotoxic Activity of the Chimeric Antibodies

This example tested the cytotoxic activity of three selected chimeric antibodies, 94C4C3, 108F3D1E9, and xi31D9B3.

Rko cells that stably expressed AMHR2 were seeded to a 96-well plate at 3000 cells per well. After overnight incubation, each diluted sample was added. After 5 days incubation, cell viability was measured by CellTiter-Glo reagent. The luciferase activity was detected by Envison.

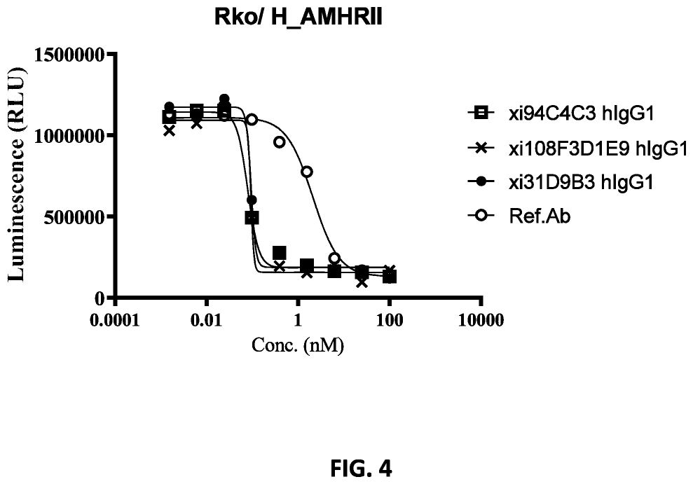

The results are presented in FIG. 4 and Table 6, which show that these chimeric antibodies have stronger endocytosis-inducing activities, as compared to Ref.Ab.

TABLE 6
Cytotoxic Activity of the Antibodies

| Antibody | IC₅₀ (nM) | Top (MFI) |
|---|---|---|
| xi94C4C3 hlgG1 | 0.08 | 1143025 |
| xi108F3D1E9 hlgG1 | ~0.09356 | 1091674 |
| xi31D9B3 hlgG1 | ~0.09405 | 1172881 |
| Ref.Ab | 2.11 | 1108735 |

### Example 6. Humanization of the Antibodies

This example prepared humanized antibodies from the murine antibodies 94C4C3, 108F3D1E9 and 31D9B3.

The variable region genes of the murine antibodies were employed to create humanized mAbs. The amino acid sequences of the VH and VL of mAb were compared against the available database of human Ig gene sequences to find the overall best-matching human germline Ig gene sequences. The CDRs of the murine antibodies were then grafted into the matched human sequences. The cDNAs were synthesized and used to produce the humanized antibodies. Certain back mutations from the murine antibodies were then introduced back to the humanized antibodies. Certain amino acids were mutated to reduce the chance of post-translational modification.

The amino acid sequences of the humanized antibodies are provided below.

### Humanized Sequences

### A. 94C4C3

TABLE 7A
Humanization of 94C4C3-VH

| | | SEQ |
|---|---|---|
| Name | | ID |
| Sequence | | NO: |
| 94C4C3 VH | DVQLQESGPGLVKPSQSLSLTCTVTGYSITSDYAWNWIRQFPGNKLEWMG | 1 |
| | YISYSGSISYNPSLKSRISITRDTSKNQFFLQLNSVTTEDTATYYCARSR | |
| | YSWFDYWGQGTLVTVSA | |
| V1 (CDR | QLQLQESGPGLVKPSETLSLTCTVSGYSITSDYAWNWIRQPPGKGLEWIG | 40 |
| grafting) | YISYSGSISYNPSLKSRVTISVDTSKNQFSLKLSSVTAADTAVYYCARSR | |
| | YSWFDYWGQGTLVTVSS | |
| V2 (with back | QLQLQESGPGLVKPSETLSLTCTVSGYSITSDYAWNWIRQPPGKGLEWMG | 41 |
| mutations) | YISYSGSISYNPSLKSRITISRDTSKNQFSLKLSSVTAADTAVYYCARSR | |
| | YSWFDYWGQGTLVTVSS | |
| V3 (with back | QVQLQESGPGLVKPSETLSLTCTVSGYSITSDYAWNWIRQFPGKGLEWMG | 42 |
| mutations) | YISYSGSISYNPSLKSRITISRDTSKNQFFLKLSSVTAADTAVYYCARSR | |
| | YSWFDYWGQGTLVTVSS | |
| V4 (with back | QVQLQESGPGLVKPSETLSLTCTVIGYSITSDYAWNWIRQFPGKKLEWMG | 43 |
| mutations) | YISYSGSISYNPSLKSRITISRDTSKNQFFLQLSSVTAADTAVYYCARSR | |
| | YSWFDYWGQGTLVTVSS | |

TABLE 7B
CDR Sequences

| CDR | Sequence | SEQ ID NO: |
|---|---|---|
| CDR-H1 | SDYAWN | 13 |
| CDR-H2 | YISYSGSISYNPSLKS | 14 |
| CDR-H3 | SRYSWFDY | 15 |

TABLE 7C
Humanization of 94C4C3-VL

| Name | Sequence | SEQ ID NO: |
|---|---|---|
| 94C4C3 VL | DIVLTQSPTSLAVSLGQRATLSCSASESVDTYGNSFMHWYQQKPGQPPK | 2 |
| | LLIYRASSLESGIPVRFSGSGSRTDFTLTINPVEAADVATYYCQQSNED | |
| | PYTFGGGTKLEIK | |
| V1 (CDR | EIVLTQSPGTLSLSPGERATLSCSASESVDTYGNSFMHWYQQKPGQAPR | 44 |
| grafting) | LLIYRASSLESGIPDRFSGSGSGTDFTLTISRLEPEDFAVYYCQQSNED | |
| | PYTFGGGTKVEIK | |
| V2 (with back | EIVLTQSPGTLSLSPGERATLSCSASESVDTYGNSFMHWYQQKPGQPPR | 45 |
| mutations) | LLIYRASSLESGIPVRFSGSGSRTDFTLTISRLEPEDFAVYYCQQSNED | |
| | PYTFGGGTKVEIK | |
| V3 (with back | EIVLTQSPGTLSLSPGERATLSCSASESVDTYGNSFMHWYQQKPGQPPR | 46 |
| mutations) | LLIYRASSLESGIPVRFSGSGSRTDFTLTISRVEAEDFAVYYCQQSNED | |
| | PYTFGGGTKLEIK | |
| V4 (with back | DIVLTQSPGTLSVSPGERATLSCSASESVDTYGNSFMHWYQQKPGQPPK | 47 |
| mutations) | LLIYRASSLESGIPVRFSGSGSRTDFTLTISRVEAEDFAVYYCQQSNED | |
| | PYTFGGGTKLEIK | |

TABLE 7D
CDR Sequences

| CDR | Sequence | SEQ ID NO: |
|---|---|---|
| CDR-L1 | SASESVDTYGNSFMH | 16 |
| CDR-L2 | RASSLES | 17 |
| CDR-L3 | QQSNEDPYT | 18 |

TABLE 7E
Humanized antibodies

| | VH | | | |
|---|---|---|---|---|
| | 94C4C3 | 94C4C3 | 94C4C3 | 94C4C3 |
| VL | VH_1 | VH_2 | VH_3 | VH_4 |
| 94C4C3 VL_1 | H1L1 | H2L1 | H3L1 | H4L1 |
| 94C4C3 VL_2 | H1L2 | H2L2 | H3L2 | H4L2 |
| 94C4C3 VL_3 | H1L3 | H2L3 | H3L3 | H4L3 |
| 94C4C3 VL_4 | H1L4 | H2L4 | H3L4 | H4L4 |

TABLE 8A
Humanization of 108F3DIE9-VH

| Name | Sequence | SEQ ID NO: |
|---|---|---|
| 108F3D1E9 | DVQLQESGPGLVKPSQSLSLTCTVTGYSITSDFAWNWIRQFPGNKLEWMG | 5 |
| VH | YINFSGSTSYNPSLKSRISITRDTSKNQFFLQLNSVTTEDTATYYCAGSR | |
| | YSWFAYWGQGTLVTVSA | |
| V1 (CDR | QLQLQESGPGLVKPSETLSLTCTVSGYSITSDFAWNWIRQPPGKGLEWIG | 48 |
| grafting, PTM | 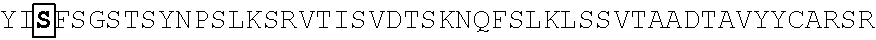 | |
| de-risked) | YSWFAYWGQGTLVTVSS | |
| V2 (with back | QLQLQESGPGLVKPSETLSLTCTVSGYSITSDFAWNWIRQPPGKGLEWMG | 49 |
| mutations) | 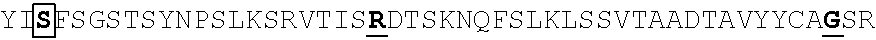 | |
| | YSWFAYWGQGTLVTVSS | |
| V3 (with back | QVQLQESGPGLVKPSETLSLTCTVSGYSITSDFAWNWIRQFPGKGLEWMG | 50 |
| mutations) | 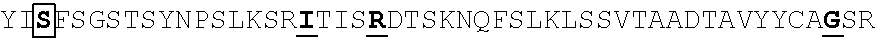 | |
| | YSWFAYWGQGTLVTVSS | |
| V4 (with back | QVQLQESGPGLVKPSETLSLTCTVSGYSITSDFAWNWIRQFPGKGLEWMG | 51 |
| mutations) | 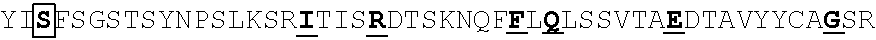 | |
| | YSWFAYWGQGTLVTVSS | |

TABLE 8B
CDR Sequences

| CDR | Sequence | SEQ ID NO: |
|---|---|---|
| CDR-H1 | SDFAWN | 20 |
| CDR-H2 | YINFSGSTSYNPSLKS | 21 |
| CDR-H2 | YISFSGSTSYNPSLKS | 33 |
| (N52S) | | |
| CDR-H3 | SRYSWFAY | 22 |

TABLE 8C
Humanization of 108F3D1E9-VL

| Name | Sequence | SEQ ID NO: |
|---|---|---|
| 108F3D1E9 | DIVLTQSPASLAVSLGQRATISCRASESVDTYGNSFMHWYQQKPGQPPK | 6 |
| VL | LLIYRASNLESGIPARFSGSGSRTDFTLTINPVEADDVATYYCQQSNED | |
| | PYTFGGGTKLEIK | |
| V1 (CDR | DIQMTQSPSSLSASVGDRVTITCRASESVDTYGNSFMHWYQQKPGKAPK | 52 |
| grafting) | LLIYRASNLESGVPSRFSGSGSGTDFTLTISSLQPEDFATYYCQQSNED | |
| | PYTFGGGTKVEIK | |
| V2 (with back | DIQLTQSPSSLSASVGDRVTITCRASESVDTYGNSFMHWYQQKPGKPPK | 53 |
| mutations) | LLIYRASNLESGIPSRFSGSGSGTDFTLTISSLQPEDFATYYCQQSNED | |
| | PYTFGGGTKVEIK | |
| V3 (with back | DIVLTQSPSSLSASVGDRVTITCRASESVDTYGNSFMHWYQQKPGQPPK | 54 |
| mutations) | LLIYRASNLESGIPARFSGSGSGTDFTLTISSVQPEDFATYYCQQSNED | |
| | PYTFGGGTKVEIK | |
| V4 (with back | DIVLTQSPSSLSVSVGDRATITCRASESVDTYGNSFMHWYQQKPGQPPK | 55 |
| mutations) | LLIYRASNLESGIPARFSGSGSRTDFTLTISSVQPEDFATYYCQQSNED | |
| | PYTFGGGTKLEIK | |

TABLE 8D
CDR Sequences

| CDR | Sequence | SEQ ID NO: |
|---|---|---|
| CDR-L1 | RASESVDTYGNSFMH | 23 |
| CDR-L2 | RASNLES | 24 |
| CDR-L3 | QQSNEDPYT | 18 |

TABLE 8E
Humanized antibodies

| | VH | | | |
|---|---|---|---|---|
| | 108F3D1E9 | 108F3D1E9 | 108F3D1E9 | 108F3D1E9 |
| VL | VH_1 | VH_2 | VH_3 | VH_4 |
| 108F3D1E9 VL_1 | H1L1 | H2L1 | H3L1 | H4L1 |
| 108F3D1E9 VL_2 | H1L2 | H2L2 | H3L2 | H4L2 |
| 108F3D1E9 VL_3 | H1L3 | H2L3 | H3L3 | H4L3 |
| 108F3D1E9 VL_4 | H1L4 | H2L4 | H3L4 | H4L4 |

TABLE 9A
Humanization of 31D9B3-VH

| Name | Sequence | SEQ ID NO: |
|---|---|---|
| 31D9B3 VH | QVKLLQSGAALVKPGASVKMSCKASGYTFTDYWVSWVKQSHGKSLEWIGE | 9 |
| | IYPKNGVSNFNEKFKGKATLTVDKSTSTAYMELRRLTSEDSAIFYCTROR | |
| | DYWGQGVMVTVSS | |
| V1 (CDR | QVQLVQSGAEVKKPGASVKVSCKASGYTFTDYWVSWVRQAPGQGLEWMGE | 56 |
| grafting) | IYPKNGVSNFNEKFKGRVTMTTDTSTSTAYMELRSLRSDDTAVYYCARQR | |
| | DYWGQGTLVTVSS | |
| V2 (with back | QVQLVQSGAEVVKPGASVKVSCKASGYTFTDYWVSWVRQAPGQGLEWIGE | 57 |
| mutations) | TYPKNGVSNFNEKFKGRVTMTVDTSTSTAYMELRSLRSDDTAVYYCTRQR | |
| | DYWGQGTLVTVSS | |
| V3 (with back | QVQLVQSGAEVKVPGASVKMSCKASGYTFTDYWVSWVRQSPGQGLEWIGE | 58 |
| mutations) | IYPKNGVSNFNEKFKGRVTLTVDKSTSTAYMELRSLRSDDTAVYYCTRQR | |
| | DYWGQGVLVTVSS | |
| V4 (with back | QVQLVQSGAEVVKPGASVKMSCKASGYTFTDYWVSWVKQSPGQGLEWIGE | 59 |
| mutations) | TYPKNGVSNFNEKFKGKATLTVDKSTSTAYMELRRLRSDDTAVFYCTRQR | |
| | DYWGQGVLVTVSS | |

TABLE 9B
CDR Sequences

| CDR | Sequence | SEQ ID NO: |
|---|---|---|
| CDR-H1 | DYWVS | 25 |
| CDR-H2 | EIYPKNGVSNFNEKFKG | 26 |
| CDR-H3 | QRDY | 27 |

TABLE 9C
Humanization of 31D9B3-VL

| Name | Sequence | SEQ ID NO: |
|---|---|---|
| 31D9B3 VL | DVVLTQTPGSLSVTLGDQASISCRSSQSLEYGDGYTYLEWYLQKPGQSP | 10 |
| | QLLIYGVSNRFSGVPDRFIGSGSGTDFTLKISRVEPEDLGVYYCFQATH | |
| | DPNTFGAGTKLELK | |
| V1 (CDR | DVVMTQSPLSLPVTLGQPASISCRSSQSLEYGDGYTYLEWFQQRPGQSP | 60 |
| grafting) | RRLIYGVSNRFSGVPDRFSGSGSGTDFTLKISRVEAEDVGVYYCFQATH | |
| | DPNTFGQGTKLEIK | |
| V2 (with back | DVVMTQSPLSLPVTLGQPASISCRSSQSLEYGDGYTYLEWYLQRPGQSP | 61 |
| mutations) | RLLIYGVSNRFSGVPDRFSGSGSGTDFTLKISRVEAEDVGVYYCFQATH | |
| | DPNTFGQGTKLEIK | |
| V3 (with back | DVVMTQSPLSLPVTLGQPASISCRSSQSLEYGDGYTYLEWYLQRPGQSP | 62 |
| mutations) | RLLIYGVSNRFSGVPDRFIGSGSGTDFTLKISRVEAEDVGVYYCFQATH | |
| | DPNTFGAGTKLEIK | |
| V4 (with back | DVVLTQSPLSLPVTLGQPASISCRSSQSLEYGDGYTYLEWYLQRPGQSP | 63 |
| mutations) | QLLIYGVSNRFSGVPDRFIGSGSGTDFTLKISRVEPEDVGVYYCFQATH | |
| | DPNTFGAGTKLEIK | |

TABLE 9D
CDR Sequences

| CDR | Sequence | SEQ ID NO: |
|---|---|---|
| CDR-L1 | RSSQSLEYGDGYTYLE | 28 |
| CDR-L2 | GVSNRFS | 29 |
| CDR-L3 | FQATHDPNT | 30 |

TABLE 9E
Humanized antibodies

| | VH | | | |
|---|---|---|---|---|
| | 31D9B3 | 31D9B3 | 31D9B3 | 31D9B3 |
| VL | VH_1 | VH_2 | VH_3 | VH_4 |
| 31D9B3 VL_1 | H1L1 | H2L1 | H3L1 | H4L1 |
| 31D9B3 VL_2 | H1L2 | H2L2 | H3L2 | H4L2 |
| 31D9B3 VL_3 | H1L3 | H2L3 | H3L3 | H4L3 |
| 31D9B3 VL_4 | H1L4 | H2L4 | H3L4 | H4L4 |

TABLE 10A
Humanization of 31D9B3-VH (PTM de-risked based on VH_V1)

| Name | Sequence | SEQ ID NO: |
|---|---|---|
| 31D9B3 VH | QVKLLQSGAALVKPGASVKMSCKASGYTFTDYWVSWVKQSHGKSLEWIGE | 9 |
| | IYPKNGVSNFNEKFKGKATLTVDKSTSTAYMELRRLTSEDSAIFYCTRQR | |
| | DYWGQGVMVTVSS | |
| VH_1.1 | QVQLVQSGAEVKKPGASVKVSCKASGYTFTDYWVSWVRQAPGQGLEWMGE | 64 |
| (N54E) | 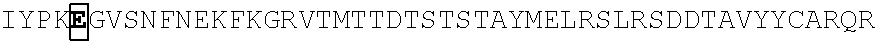 | |
| | DYWGQGTLVTVSS | |
| VH_1.2 | QVQLVQSGAEVKKPGASVKVSCKASGYTFTDYWVSWVRQAPGQGLEWMGE | 65 |
| (G55D) | 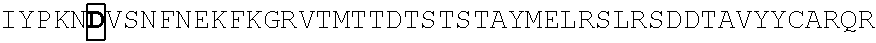 | |
| | DYWGQGTLVTVSS | |
| VH_1.3 | QVQLVQSGAEVKKPGASVKVSCKASGYTFTDYWVSWVRQAPGQGLEWMGE | 66 |
| (G55Q) | 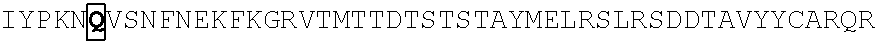 | |
| | DYWGQGTLVTVSS | |

TABLE 10B
CDR Sequences

| | | SEQ |
|---|---|---|
| | | ID |
| CDR | Sequence | NO: |
| CDR-H1 | DYWVS | 25 |
| CDR-H2 | EIYPKNGVSNFNEKFKG | 26 |
| CDR-H2 (N54E) | EIYPKEGVSNFNEKFKG | 34 |
| CDR-H2 (G55D) | EIYPKNDVSNFNEKFKG | 35 |
| CDR-H2 (G55Q) | EIYPKNQVSNFNEKFKG | 36 |
| CDR-H3 | QRDY | 27 |

TABLE 10C
Humanization of 31D9B3-VL (based on VL_V2)

| Name | Sequence | SEQ ID NO: |
|---|---|---|
| 31D9B3 VL | DVVLTQTPGSLSVTLGDQASISCRSSOSLEYGDGYTYLEWYLQKPGQSP | 10 |
| | QLLIYGVSNRFSGVPDRFIGSGSGTDFTLKISRVEPEDLGVYYCFQATH | |
| | DPNTFGAGTKLELK | |
| VL_2.1 | 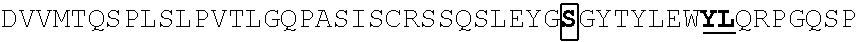 | 67 |
| (D(30C)S) | RLLIYGVSNRFSGVPDRFSGSGSGTDFTLKISRVEAEDVGVYYCFQATH | |
| | DPNTFGQGTKLEIK | |
| VL_2.2 | 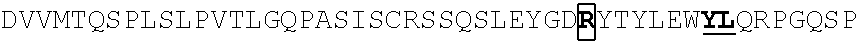 | 68 |
| (G(30D)R) | RLLIYGVSNRFSGVPDRFSGSGSGTDFTLKISRVEAEDVGVYYCFQATH | |
| | DPNTFGQGTKLEIK | |
| VL_2.3 | 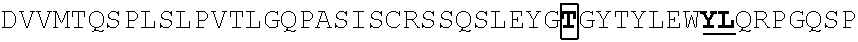 | 69 |
| (D(30C)T) | RLLIYGVSNRFSGVPDRFSGSGSGTDFTLKISRVEAEDVGVYYCFQATH | |
| | DPNTFGQGTKLEIK | |

TABLE 10D
CDR Sequences

| CDR | Sequence | SEQ ID NO: |
|---|---|---|
| CDR-L1 | RSSQSLEYGDGYTYLE | 28 |
| CDR-L1 (D(30C)S) | RSSQSLEYGSGYTYLE | 37 |
| CDR-L1 (G(30D)R) | RSSQSLEYGDRYTYLE | 38 |
| CDR-L1 (D(30C)T) | RSSQSLEYGTGYTYLE | 39 |
| CDR-L2 | GVSNRFS | 29 |
| CDR-L3 | FQATHDPNT | 30 |

TABLE 10E
Humanized antibodies - PTM de-risked

| | VL | | | |
|---|---|---|---|---|
| VH | VL_2 | VL_2.1 | VL_2.2 | VL_2.3 |
| VH_1 | VH_1-VL_2 | VH_1-VL_2.1 | VH_1-VL_2.2 | VH_1-VL_2.3 |
| VH_1.1 | VH_1.1-VL_2 | VH_1.1-VL_2.1 | VH_1.1-VL_2.2 | VH_1.1-VL_2.3 |
| VH_1.2 | VH_1.2-VL_2 | VH_1.2-VL_2.1 | VH_1.2-VL_2.2 | VH_1.2-VL_2.3 |
| VH_1.3 | VH_1.3-VL_2 | VH_1.3-VL_2.1 | VH_1.3-VL_2.2 | VH_1.3-VL_2.3 |

### Example 7. Binding Activity of the Humanized Antibodies

This example tested the binding affinities of the humanized antibodies in binding to the human AMHRII protein expressed on cells.

CHO-K1 cells over-expressed AMHR2 were harvested from flasks. 100 μl of 1×10⁶ cells/ml of cells were incubated with the humanized antibodies in 3-fold serial dilutions starting from 300 nM to 0.001 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

The results of the study are presented in FIG. 5A-H, and Tables 11A-H. Most of the humanized antibodies exhibited high binding affinity to AMHRII.

TABLE 11A
Binding Affinity of Humanized 94C4C3

| Antibody | EC₅₀ (nM) | Top (MFI) |
|---|---|---|
| xi94C4C3 hlgG1 | 1.21 | 54931 |
| 94C4C3 H1L1 hlgG1 | 1.70 | 53007 |
| 94C4C3 H1L2 hlgG1 | 1.46 | 49606 |
| 94C4C3 H1L3 hlgG1 | 1.45 | 50159 |
| 94C4C3 H1L4 hlgG1 | 1.21 | 47245 |
| 94C4C3 H2L1 hlgG1 | 1.31 | 52776 |
| 94C4C3 H2L2 hlgG1 | 1.42 | 56608 |
| 94C4C3 H2L3 hlgG1 | 1.32 | 55782 |
| 94C4C3 H2L4 hlgG1 | 1.01 | 52461 |

TABLE 11B
Binding Affinity of Humanized 94C4C3 (cont'd)

| Antibody | EC₅₀ (nM) | Top (MFI) |
|---|---|---|
| xi94C4C3 hlgG1 | 1.21 | 54931 |
| 94C4C3 H3L1 hlgG1 | 1.32 | 51046 |
| 94C4C3 H3L2 hlgG1 | 1.23 | 47680 |
| 94C4C3 H3L3 hlgG1 | 1.03 | 46276 |
| 94C4C3 H3L4 hlgG1 | 1.35 | 48053 |
| 94C4C3 H4L1 hlgG1 | 1.12 | 49517 |
| 94C4C3 H4L2 hlgG1 | 1.02 | 48693 |
| 94C4C3 H4L3 hlgG1 | 1.16 | 51095 |
| 94C4C3 H4L4 hlgG1 | 1.09 | 57881 |

TABLE 11C
Binding Affinity of Humanized 31D9B3

| Antibody | EC₅₀ (nM) | Top (MFI) |
|---|---|---|
| xi31D9B3 hlgG1 | 0.88 | 35229 |
| 31D9B3 H1L1 hlgG1 | 89.11 | 8935 |
| 31D9B3 H1L2 hlgG1 | 1.49 | 36220 |
| 31D9B3 H1L3 hlgG1 | 1.36 | 34685 |
| 31D9B3 H1L4 hlgG1 | 1.42 | 34118 |
| 31D9B3 H2L1 hlgG1 | 22.54 | 19110 |
| 31D9B3 H2L2 hlgG1 | 1.58 | 37948 |
| 31D9B3 H2L3 hlgG1 | 1.28 | 36690 |
| 31D9B3 H2L4 hlgG1 | 1.67 | 39230 |

TABLE 11D
Binding Affinity of Humanized 31D9B3 (cont'd)

| Antibody | EC₅₀ (nM) | Top (MFI) |
|---|---|---|
| xi31D9B3 hlgG1 | 0.88 | 35229 |
| 31D9B3 H3L1 hlgG1 | 3.01 | 19596 |
| 31D9B3 H3L2 hlgG1 | 1.05 | 37903 |
| 31D9B3 H3L3 hlgG1 | 1.30 | 37974 |
| 31D9B3 H3L4 hlgG1 | 1.01 | 36441 |
| 31D9B3 H4L1 hlgG1 | 2.18 | 16060 |
| 31D9B3 H4L2 hlgG1 | 0.84 | 36162 |
| 31D9B3 H4L3 hlgG1 | 0.83 | 36001 |
| 31D9B3 H4L4 hlgG1 | 0.75 | 37239 |

TABLE 11E
Binding Affinity of Humanized 108F3D1E9

| Antibody | EC₅₀ (nM) | Top (MFI) |
|---|---|---|
| xi108F3D1E9 hlgG1 | 1.05 | 74408 |
| 108F3D1E9 H1L1 hlgG1 | 0.74 | 65421 |
| 108F3D1E9 H1L2 hlgG1 | 0.82 | 64640 |
| 108F3D1E9 H1L3 hlgG1 | 0.82 | 66672 |
| 108F3D1E9 H1L4 hlgG1 | 0.88 | 70941 |
| 108F3D1E9 H2L1 hlgG1 | 0.55 | 64435 |
| 108F3D1E9 H2L2 hlgG1 | 0.68 | 67878 |
| 108F3D1E9 H2L3 hlgG1 | 0.74 | 67290 |
| 108F3D1E9 H2L4 hlgG1 | 0.65 | 70623 |

TABLE 11F
Binding Affinity of Humanized 108F3D1E9 (cont'd)

| Antibody | EC₅₀ (nM) | Top (MFI) |
|---|---|---|
| xi108F3D1E9 hlgG1 | 1.05 | 74408 |
| 108F3D1E9 H3L1 hlgG1 | 0.74 | 65421 |
| 108F3D1E9 H3L2 hlgG1 | 0.82 | 64640 |
| 108F3D1E9 H3L3 hlgG1 | 0.82 | 66672 |
| 108F3D1E9 H3L4 hlgG1 | 0.88 | 70941 |
| 108F3D1E9 H4L1 hlgG1 | 0.55 | 64435 |
| 108F3D1E9 H4L2 hlgG1 | 0.68 | 67878 |
| 108F3D1E9 H4L3 hlgG1 | 0.74 | 67290 |
| 108F3D1E9 H4L4 hlgG1 | 0.65 | 70623 |

TABLE 11G
Binding Affinity of Humanized 31D9B3 (PTM de-risked)

| Antibody | EC₅₀ (nM) | Top (MFI) |
|---|---|---|
| 31D9B3 hlgG1 | 0.8806 | 26220 |
| 31D9B3 VH1-VL2 hlgG1 | 1.056 | 26686 |
| 31D9B3 VH1-VL2.1 hlgG1 | 5.517 | 10390 |
| 31D9B3 VH1-VL2.2 hlgG1 | 0.4851 | 22399 |
| 31D9B3 VH1-VL2.3 hlgG1 | 0.3309 | 12510 |
| 31D9B3 VH1.1-VL2 hlgG1 | 0.8752 | 16825 |
| 31D9B3 VH1.1-VL2.1 hlgG1 | 89.7 | 2174 |
| 31D9B3 VH1.1-VL2.2 hlgG1 | 0.6255 | 14536 |
| 31D9B3 VH1.1-VL2.3 hlgG1 | 145.1 | 6581 |

TABLE 11H
Binding Affinity of Humanized 31D9B3 (PTM de-risked; cont'd)

| Antibody | EC₅₀ (nM) | Top (MFI) |
|---|---|---|
| 31D9B3 hlgG1 | 0.8806 | 26220 |
| 31D9B3 VH1.2-VL2 hlgG1 | 1.507 | 26813 |
| 31D9B3 VH1.2-VL2.1 hlgG1 | 5.684 | 10610 |
| 31D9B3 VH1.2-VL2.2 hlgG1 | 0.8557 | 22028 |
| 31D9B3 VH1.2-VL2.3 hlgG1 | 0.6167 | 12927 |
| 31D9B3 VH1.3-VL2 hlgG1 | 1.175 | 28014 |
| 31D9B3 VH1.3-VL2.1 hlgG1 | 1.197 | 10927 |
| 31D9B3 VH1.3-VL2.2 hlgG1 | 0.7032 | 24134 |
| 31D9B3 VH1.3-VL2.3 hlgG1 | 0.3799 | 12693 |

### Example 8. FACS Binding Activity of the Humanized Antibodies

This example further tested the binding affinities of some of the humanized/PTM de-risked antibodies with FACS.

Rko cells over-expressing AMHR2 were harvested from flasks. 100 μl of 1×10⁶ cells/ml of cells were incubated with primary PTM antibodies in 3-fold serial dilutions starting from 300 nM to 0.001 nM for 30 minutes on ice. After being washed with 200 μl of FACS buffer twice, cells were incubated with secondary antibody for 30 minutes on ice. Cells were washed with 200 μl of FACS buffer twice and analyzed by FACS.

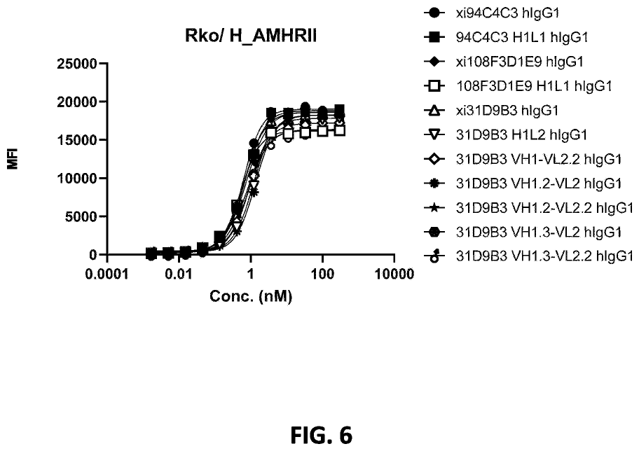

The results of the study are presented in FIG. 6, and Tables 12. The results show that the PTM antibodies can bind to human AMHR2 potently.

TABLE 12
FACS Binding Affinity of PTM de-risked Antibodies

| Antibody | EC₅₀ (nM) | Top (MFI) |
|---|---|---|
| xi94C4C3 hlgG1 | 0.6267 | 19032 |
| 94C4C3 H1L1 hlgG1 | 0.6946 | 18873 |
| xi108F3D1E9 hlgG1 | 0.7562 | 18704 |
| 108F3D1E9 H1L1 hlgG1 | 0.5283 | 16186 |
| xi31D9B3 hlgG1 | 0.6575 | 18633 |
| 31D9B3 H1L2 hlgG1 | 1.205 | 18269 |
| 31D9B3 VH1-VL2.2 hlgG1 | 0.915 | 17219 |
| 31D9B3 VH1.2-VL2 hlgG1 | 1.35 | 18243 |
| 31D9B3 VH1.2-VL2.2 hlgG1 | 0.8779 | 16281 |
| 31D9B3 VH1.3-VL2 hlgG1 | 0.9756 | 17855 |
| 31D9B3 VH1.3-VL2.2 hlgG1 | 0.7835 | 16397 |

### Example 9. Further Testing of Selected Humanized Antibodies

This example further tested the activities of humanized antibodies 94C4C3 H1L1, 108F3D1E9 H1L1, and 31D9B3 VH1.3-VL2.2.

The first experiment examined the binding affinity of these antibodies to AMHRII expressed on CHO-K1 and Rko cells.

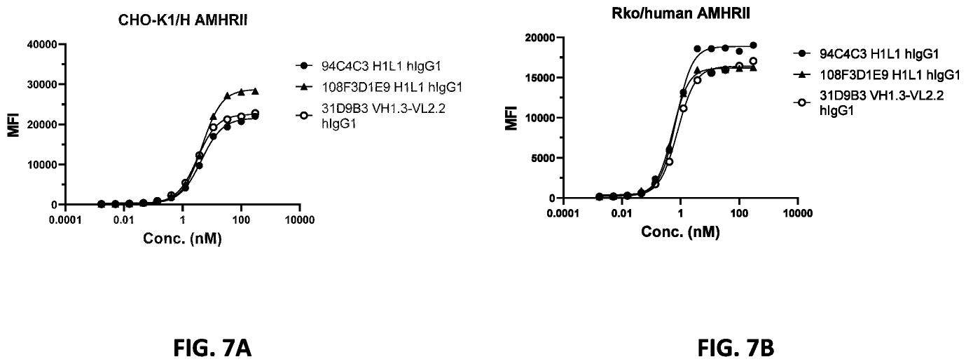

CHO-K1 and Rko cells that stably expressed AMHR2 were harvested from flasks. 100 μl of 1×10⁶ cells/ml of cells were incubated with primary humanized antibodies in 3-fold serial dilutions starting from 300 nM to 0.002 nM for 30 minutes on ice. After being washed with 200 μl of FACS buffer twice, cells were incubated with secondary antibody for 30 minutes on ice. Cells were washed with 200 μl of FACS buffer twice and analyzed by FACS. The results of the study (FIG. 7A-B) showed that the humanized antibodies can bind to human AMHR2 with high affinity.

The second experiment examined ADCC of these humanized antibodies.

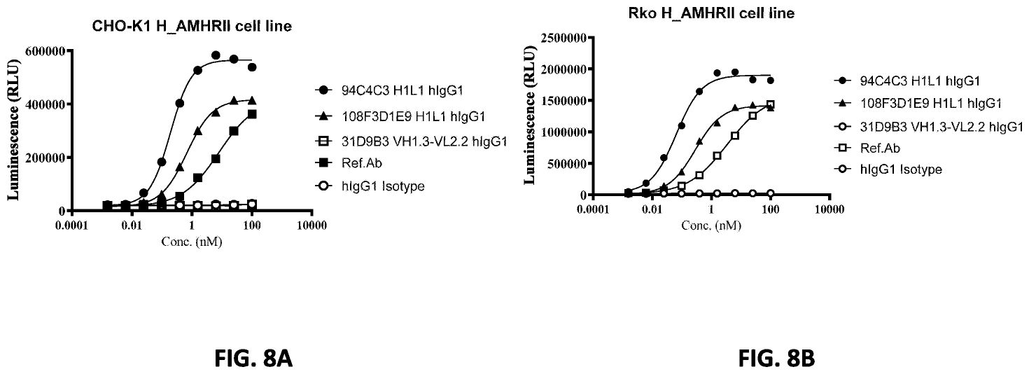

The ADCC Reporter Bioassay (see Example 3) was used for this purpose. Serial dilutions of AMHR2 humanized monoclonal antibody were incubated for 6 hours of induction at 37° C. with engineered Jurkat effector cells (ADCC Bioassay Effector Cells), with ADCC Bioassay Target Cells (expressing AMHR2). Luciferase activity was quantified using Bio-Glo™ Reagent. The results (FIG. 8A-B) show that these humanized antibodies have stronger ADCC activities, as compared to reference antibody Ref.Ab.

The third experiment examined the ability to induce endocytosis by these humanized antibodies.

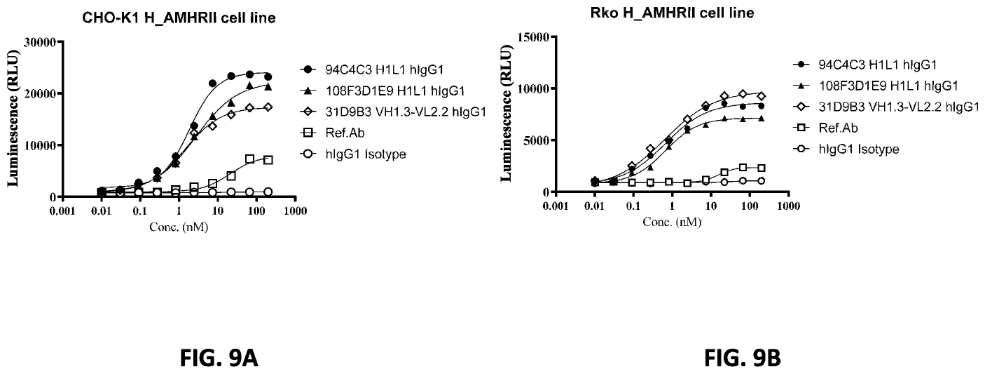

The method uses a new hydrophilic and bright pH sensor dye (pHAb dye), as described in Example 4. Serial dilutions of AMHR2 humanized monoclonal antibody were incubated for 24 hours at 37° C. Luciferase activity was detected. The results (FIG. 9A-B) show that these humanized antibodies have stronger endocytosis activities, as compared to Ref.Ab.

The fourth experiment examined whether the humanized antibodies could cross-react to the cyno, rat, and mouse AMHRII proteins.

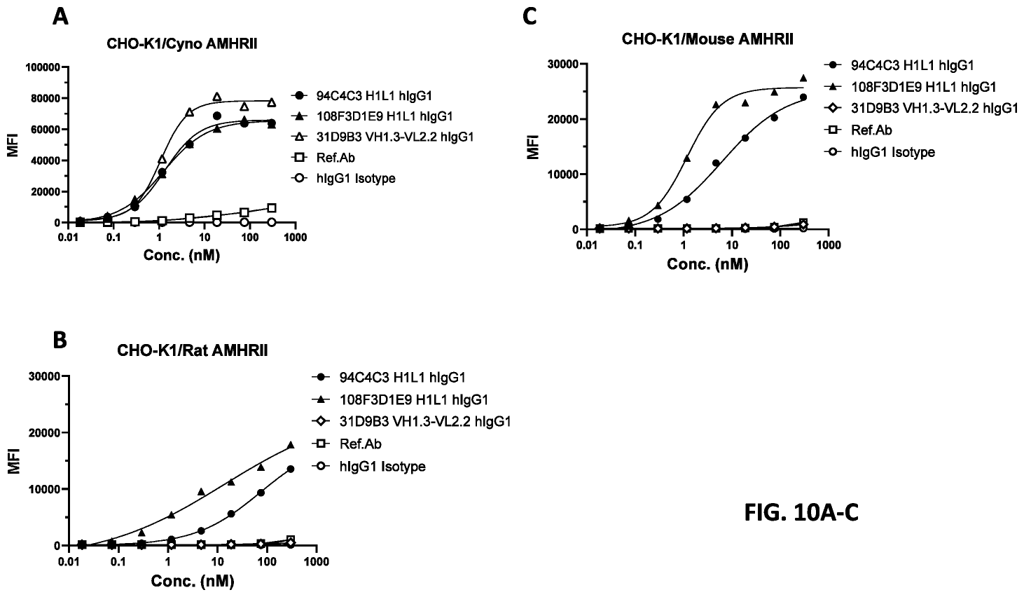

To evaluate these antibodies' cross reactivities to cyno, rat and mouse AMHR2, CHO-K1 cells over-expressing cyno, rat and mouse AMHR2, respectively, were harvested from flasks. 100 μl of 1×10⁶ cells/ml of cells were incubated with primary antibodies in 4-fold serial dilutions starting from 300 nM to 0.018 nM for 30 minutes on ice. After being washed with 200 μl of FACS buffer twice, cells were incubated with secondary antibody for 30 minutes on ice. Cells were washed with 200 μl of FACS buffer twice and analyzed by FACS. The results (FIG. 10A-C, summarized in Table 13) of the study showed that the humanized antibodies can bind to cyno, rat and mouse AMHR2 by flow cytometry.

TABLE 13
FACS Binding Affinity of PTM de-risked Antibodies

| | Mouse | Rat | Cyno |
|---|---|---|---|
| 94C4C3 H1L1 hlgG1 | √ | √ | √ |
| 108F3D1E9 H1L1 hlgG1 | √ | √ | √ |
| 31D9B3 VH1.3-VL2.2 hlgG1 | x | x | √ |
| Ref.Ab | x | x | x |

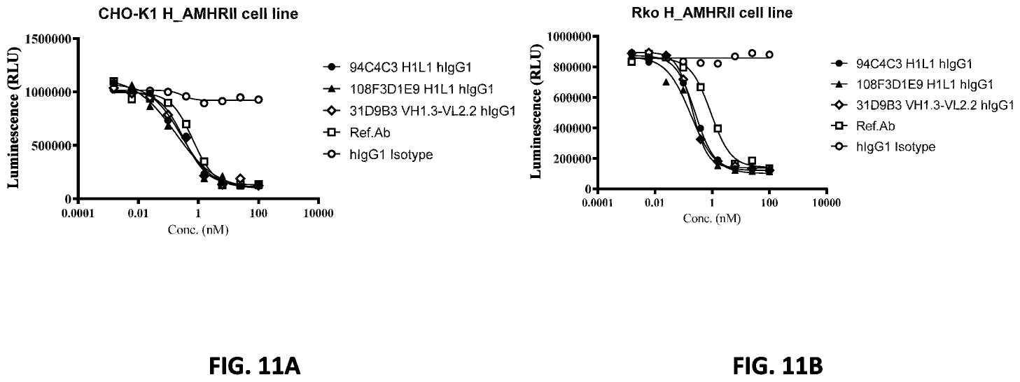

The fifth experiment examined the cytotoxic activities of the humanized antibodies. Like in Example 5, CHO-K1 and Rko cells that stably expressed AMHR2 were seeded to a 96-well plate at 3000 cells per well. After overnight incubation, each diluted sample was added. After 5 days incubation, cell viability was measured by CellTiter-Glo reagent. The luciferase activity was detected by Envison. The results (FIG. 11A-B) show that these chimeric antibodies have very strong cytotoxic activities.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof having binding specificity to a human AMHRII (anti-Mullerian hormone receptor type 2) protein, comprising a heavy chain variable region (VH) comprising a VH CDR1, VH CDR2, and VH CDR3, and a light chain variable region (VL) comprising a VL CDR1, VL CDR2 and VL CDR3, wherein:
(a) the VH CDR1 includes the amino acid sequence of SEQ ID NO:70;
the VH CDR2 includes the amino acid sequence of SEQ ID NO: 71;
the VH CDR3 includes the amino acid sequence of SEQ ID NO:72;
the VL CDR1 includes the amino acid sequence of SEQ ID NO:73;
the VL CDR2 includes the amino acid sequence of SEQ ID NO:74; and
the VL CDR3 includes the amino acid sequence of SEQ ID NO:18, or
(b) the VH CDR1 includes the amino acid sequence of SEQ ID NO:25;
the VH CDR2 includes the amino acid sequence of SEQ ID NO:75;
the VH CDR3 includes the amino acid sequence of SEQ ID NO:27;
the VL CDR1 includes the amino acid sequence of SEQ ID NO:76;
the VL CDR2 includes the amino acid sequence of SEQ ID NO:29; and
the VL CDR3 includes the amino acid sequence of SEQ ID NO:30.

2. The antibody or antigen-binding fragment thereof of claim 1, wherein:
(a) the VH CDR1 includes the amino acid sequence of SEQ ID NO:13 or 20;
the VH CDR2 includes the amino acid sequence of SEQ ID NO:14, 19, 21 or 33;
the VH CDR3 includes the amino acid sequence of SEQ ID NO:15 or 22;
the VL CDR1 includes the amino acid sequence of SEQ ID NO:16 or 23;
the VL CDR2 includes the amino acid sequence of SEQ ID NO:17 or 24; and
the VL CDR3 includes the amino acid sequence of SEQ ID NO:18, or
(b) the VH CDR1 includes the amino acid sequence of SEQ ID NO:25;
the VH CDR2 includes the amino acid sequence of SEQ ID NO:26, 31, 34, 35 or 36;
the VH CDR3 includes the amino acid sequence of SEQ ID NO:27;
the VL CDR1 includes the amino acid sequence of SEQ ID NO:28, 32, 37, 38 or 39;
the VL CDR2 includes the amino acid sequence of SEQ ID NO:29; and
the VL CDR3 includes the amino acid sequence of SEQ ID NO:30.

3. The antibody or antigen-binding fragment thereof of claim 2, wherein:
the VH CDR1 comprises the amino acid sequence of SEQ ID NO:13, the VH CDR2 comprises the amino acid sequence of SEQ ID NO:14, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18;
the VH CDR1 comprises the amino acid sequence of SEQ ID NO:20, the VH CDR2 comprises the amino acid sequence of SEQ ID NO:21 or 33, the VH CDR3 comprises the amino acid sequence of SEQ ID NO:22, the VL CDR1 comprises the amino acid sequence of SEQ ID NO:23, the VL CDR2 comprises the amino acid sequence of SEQ ID NO:24, and the VL CDR3 comprises the amino acid sequence of SEQ ID NO:18;
the VH CDR1 comprises the amino acid sequence of SEQ ID NO:25, the VH CDR2 comprises the amino acid sequence of SEQ ID NO:26, 34, 35 or 36, the VH CDR3 comprises the amino acid sequence of SEQ ID NO:27, the VL CDR1 comprises the amino acid sequence of SEQ ID NO:28, 37, 38 or 39, the VL CDR2 comprises the amino acid sequence of SEQ ID NO:29, and the VL CDR3 comprises the amino acid sequence of SEQ ID NO:30;
the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18;
the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:23, the VL CDR2 comprises the amino acid sequence of SEQ ID NO:17, and the VL CDR3 comprises the amino acid sequence of SEQ ID NO:18; or
the VH CDR1 comprises the amino acid sequence of SEQ ID NO:25, the VH CDR2 comprises the amino acid sequence of SEQ ID NO:31, the VH CDR3 comprises the amino acid sequence of SEQ ID NO:27, the VL CDR1 comprises the amino acid sequence of SEQ ID NO:32, the VL CDR2 comprises the amino acid sequence of SEQ ID NO:29, and the VL CDR3 comprises the amino acid sequence of SEQ ID NO:30.

4. The antibody or fragment of claim 3, wherein the VH CDR1 comprises the amino acid sequence of SEQ ID NO:13, the VH CDR2 comprises the amino acid sequence of SEQ ID NO:14, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18.

5. The antibody or fragment of claim 4, wherein the VH comprises an amino acid sequence selected from the group consisting of SEQ ID NO:1 and 40-43, and the VL comprises an amino acid sequence selected from the group consisting of SEQ ID NO:2 and 44-47.

6. The antibody or fragment of claim 4, wherein the VH comprises the amino acid sequence of SEQ ID NO:40, and the VL comprises the amino acid sequence of SEQ ID NO:44.

7. The antibody or fragment of claim 3, wherein the VH CDR1 comprises the amino acid sequence of SEQ ID NO:20, the VH CDR2 comprises the amino acid sequence of SEQ ID NO:21 or 33, the VH CDR3 comprises the amino acid sequence of SEQ ID NO:22, the VL CDR1 comprises the amino acid sequence of SEQ ID NO:23, the VL CDR2 comprises the amino acid sequence of SEQ ID NO:24, and the VL CDR3 comprises the amino acid sequence of SEQ ID NO:18.

8. The antibody or fragment of claim 7, wherein the VH CDR1 comprises the amino acid sequence of SEQ ID NO:20, the VH CDR2 comprises the amino acid sequence of SEQ ID NO:33, the VH CDR3 comprises the amino acid sequence of SEQ ID NO:22, the VL CDR1 comprises the amino acid sequence of SEQ ID NO:23, the VL CDR2 comprises the amino acid sequence of SEQ ID NO:24, and the VL CDR3 comprises the amino acid sequence of SEQ ID NO:18.

9. The antibody or fragment of claim 7, wherein the VH comprises an amino acid sequence selected from the group consisting of SEQ ID NO:5 and 48-51, and the VL comprises an amino acid sequence selected from the group consisting of SEQ ID NO:6 and 52-55.

10. The antibody or fragment of claim 7, wherein the VH comprises the amino acid sequence of SEQ ID NO:48, and the VL comprises the amino acid sequence of SEQ ID NO:52.

11. The antibody or fragment of claim 3, wherein the VH CDR1 comprises the amino acid sequence of SEQ ID NO:25, the VH CDR2 comprises the amino acid sequence of SEQ ID NO:26, 34, 35 or 36, the VH CDR3 comprises the amino acid sequence of SEQ ID NO:27, the VL CDR1 comprises the amino acid sequence of SEQ ID NO:28, 37, 38 or 39, the VL CDR2 comprises the amino acid sequence of SEQ ID NO:29, and the VL CDR3 comprises the amino acid sequence of SEQ ID NO:30.

12. The antibody or fragment of claim 11, wherein the VH CDR1 comprises the amino acid sequence of SEQ ID NO:25, the VH CDR2 comprises the amino acid sequence of SEQ ID NO:36, the VH CDR3 comprises the amino acid sequence of SEQ ID NO:27, the VL CDR1 comprises the amino acid sequence of SEQ ID NO:38, the VL CDR2 comprises the amino acid sequence of SEQ ID NO:29, and the VL CDR3 comprises the amino acid sequence of SEQ ID NO:30.

13. The antibody or fragment of claim 11, wherein the VH comprises an amino acid sequence selected from the group consisting of SEQ ID NO:9, 56-59 and 64-66, and the VL comprises an amino acid sequence selected from the group consisting of SEQ ID NO:10, 60-63 and 67-69.

14. The antibody or fragment of claim 11, wherein the VH comprises the amino acid sequence of SEQ ID NO:66, and the VL comprises the amino acid sequence of SEQ ID NO:68.

15. The antibody or fragment of claim 3, wherein the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:17, and the VL CDR3 comprises the amino acid sequence of SEQ ID NO:18.

16. The antibody or fragment of claim 15, wherein the VH comprises the amino acid sequence of SEQ ID NO:3, and the VL comprises the amino acid sequence of SEQ ID NO:4.

17. The antibody or fragment of claim 3, wherein the VH CDR1 comprises the amino acid sequence of SEQ ID NO:13, the VH CDR2 comprises the amino acid sequence of SEQ ID NO:19, the VH CDR3 comprises the amino acid sequence of SEQ ID NO:15, the VL CDR1 comprises the amino acid sequence of SEQ ID NO:23, the VL CDR2 comprises the amino acid sequence of SEQ ID NO:17, and the VL CDR3 comprises the amino acid sequence of SEQ ID NO:18.

18. The antibody or fragment of claim 17, wherein the VH comprises the amino acid sequence of SEQ ID NO:7, and the VL comprises the amino acid sequence of SEQ ID NO:8.

19. The antibody or fragment of claim 3, wherein the VH CDR1 comprises the amino acid sequence of SEQ ID NO:25, the VH CDR2 comprises the amino acid sequence of SEQ ID NO:31, the VH CDR3 comprises the amino acid sequence of SEQ ID NO:27, the VL CDR1 comprises the amino acid sequence of SEQ ID NO:32, the VL CDR2 comprises the amino acid sequence of SEQ ID NO:29, and the VL CDR3 comprises the amino acid sequence of SEQ ID NO:30.

20. The antibody or fragment of claim 19, wherein the VH comprises the amino acid sequence of SEQ ID NO:11, and the VL comprises the amino acid sequence of SEQ ID NO:12.

21. A bispecific antibody comprising the antibody or fragment of claim 1 and a second antibody or antigen-binding fragment having binding specificity to a target antigen that is not AMHRII.

22. One or more polynucleotide(s) encoding the antibody or fragment of claim 1.

23. A cell comprising the polynucleotide(s) of claim 22.

24. (canceled)

25. A method of treating cancer in a patient in need thereof, comprising administering to the patient an effective amount of the antibody or fragment of claim 1.

26. The method of claim 25, wherein the cancer is characterized with expression of the AMHRII protein.

27. The method of claim 25, wherein the cancer is selected from the group consisting of is a solid tumor. In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

28. The method of claim 27, wherein the cancer is ovarian cancer or colorectal cancer.

29. A method for killing a cell expressing the AMHRII protein, comprising contacting the cell with the antibody or fragment of claim 1.

30-32. (canceled)


SEQUENCE LISTING (76 sequences)

SEQ ID NO: 1 - protein, 117 aa, synthetic construct
DVQLQESGPGLVKPSQSLSLTCTVTGYSITSDYAWNWIRQFPGNKLEWMGYISYSGSISYNPSLKSRISITRDTSKNQFFLQLNSVTTEDTATYYCARSRYSWFDYWGQGTLVTVSA

SEQ ID NO: 2 - protein, 111 aa, synthetic construct
DIVLTQSPTSLAVSLGQRATLSCSASESVDTYGNSFMHWYQQKPGQPPKLLIYRASSLESGIPVRFSGSGSRTDFTLTINPVEAADVATYYCQQSNEDPYTFGGGTKLEIK

SEQ ID NO: 3 - protein, 117 aa, synthetic construct
DVQLQESGPGLVKPSQSLSLTCTVTGYSITSDYAWNWIRQFPGNKLEWMGYISYSGSTSYNPSLKSRISITRDTSKNQFFLQLNSVTTEDTATYYCARSRYSWFDYWGQGTLVTVSA

SEQ ID NO: 4 - protein, 111 aa, synthetic construct
DIVLTQSPTSLAVSLGQRATLSCSASESVDTYGNSFMHWYQQKPGQSPKLLIYRASSLESGIPARFSGSGSRTDFTLTINPVEAADVATYYCQQSNEDPYTFGGGTKLEIK

SEQ ID NO: 5 - protein, 117 aa, synthetic construct
DVQLQESGPGLVKPSQSLSLTCTVTGYSITSDFAWNWIRQFPGNKLEWMGYINFSGSTSYNPSLKSRISITRDTSKNQFFLQLNSVTTEDTATYYCAGSRYSWFAYWGQGTLVTVSA

SEQ ID NO: 6 - protein, 111 aa, synthetic construct
DIVLTQSPASLAVSLGQRATISCRASESVDTYGNSFMHWYQQKPGQPPKLLIYRASNLESGIPARFSGSGSRTDFTLTINPVEADDVATYYCQQSNEDPYTFGGGTKLEIK

SEQ ID NO: 7 - protein, 117 aa, synthetic construct; identical to SEQ ID NO: 3

SEQ ID NO: 8 - protein, 111 aa, synthetic construct
DIVLTQSPASLAVSLGQRATLSCRASESVDTYGNSFMHWYQQKPGQSPKLLIYRASSLESGIPARFSGSGSRTDFTLTINPVEAADVATYYCQQSNEDPYTFGGGTKLEIK

SEQ ID NO: 9 - protein, 113 aa, synthetic construct
QVKLLQSGAALVKPGASVKMSCKASGYTFTDYWVSWVKQSHGKSLEWIGEIYPKNGVSNFNEKFKGKATLTVDKSTSTAYMELRRLTSEDSAIFYCTRQRDYWGQGVMVTVSS

SEQ ID NO: 10 - protein, 112 aa, synthetic construct
DVVLTQTPGSLSVTLGDQASISCRSSQSLEYGDGYTYLEWYLQKPGQSPQLLIYGVSNRFSGVPDRFIGSGSGTDFTLKISRVEPEDLGVYYCFQATHDPNTFGAGTKLELK

SEQ ID NO: 11 - protein, 113 aa, synthetic construct
QVRLLQSGAALVKPGASAKMSCKASGYTFTDYWVSWVKQSHGKSLEWIGEIYPDNGITNFNEKFKGKATLTVDKSTRTAYMELSRLTSEDSAIYYCTRQRDYWGQGVMVTVSS

SEQ ID NO: 12 - protein, 112 aa, synthetic construct
DVVLTQTPGSLSVTLGDQVSISCRSSQSLKYGDGYTYLEWYLQKPGQSPQLLFYGVSNRFSGVPDRFIGSGSGTDFTLKISRVEPEDLGVYYCFQATHDPNTFGTGTKLDLK

SEQ ID NO: 13 - protein, 6 aa, synthetic construct
SDYAWN

SEQ ID NO: 14 - protein, 16 aa, synthetic construct
YISYSGSISYNPSLKS

SEQ ID NO: 15 - protein, 8 aa, synthetic construct
SRYSWFDY

SEQ ID NO: 16 - protein, 15 aa, synthetic construct
SASESVDTYGNSFMH

SEQ ID NO: 17 - protein, 7 aa, synthetic construct
RASSLES

SEQ ID NO: 18 - protein, 9 aa, synthetic construct
QQSNEDPYT

SEQ ID NO: 19 - protein, 16 aa, synthetic construct
YISYSGSTSYNPSLKS

SEQ ID NO: 20 - protein, 6 aa, synthetic construct
SDFAWN

SEQ ID NO: 21 - protein, 16 aa, synthetic construct
YINFSGSTSYNPSLKS

SEQ ID NO: 22 - protein, 8 aa, synthetic construct
SRYSWFAY

SEQ ID NO: 23 - protein, 15 aa, synthetic construct
RASESVDTYGNSFMH

SEQ ID NO: 24 - protein, 7 aa, synthetic construct
RASNLES

SEQ ID NO: 25 - protein, 5 aa, synthetic construct
DYWVS

SEQ ID NO: 26 - protein, 17 aa, synthetic construct
EIYPKNGVSNFNEKFKG

SEQ ID NO: 27 - protein, 4 aa, synthetic construct
QRDY

SEQ ID NO: 28 - protein, 16 aa, synthetic construct
RSSQSLEYGDGYTYLE

SEQ ID NO: 29 - protein, 7 aa, synthetic construct
GVSNRFS

SEQ ID NO: 30 - protein, 9 aa, synthetic construct
FQATHDPNT

SEQ ID NO: 31 - protein, 17 aa, synthetic construct
EIYPDNGITNFNEKFKG

SEQ ID NO: 32 - protein, 16 aa, synthetic construct
RSSQSLKYGDGYTYLE

SEQ ID NO: 33 - protein, 16 aa, synthetic construct
YISFSGSTSYNPSLKS

SEQ ID NO: 34 - protein, 17 aa, synthetic construct
EIYPKEGVSNFNEKFKG

SEQ ID NO: 35 - protein, 17 aa, synthetic construct
EIYPKNDVSNFNEKFKG

SEQ ID NO: 36 - protein, 17 aa, synthetic construct
EIYPKNQVSNFNEKFKG

SEQ ID NO: 37 - protein, 16 aa, synthetic construct
RSSQSLEYGSGYTYLE

SEQ ID NO: 38 - protein, 16 aa, synthetic construct
RSSQSLEYGDRYTYLE

SEQ ID NO: 39 - protein, 16 aa, synthetic construct
RSSQSLEYGTGYTYLE

SEQ ID NO: 40 - protein, 117 aa, synthetic construct
QLQLQESGPGLVKPSETLSLTCTVSGYSITSDYAWNWIRQPPGKGLEWIGYISYSGSISYNPSLKSRVTISVDTSKNQFSLKLSSVTAADTAVYYCARSRYSWFDYWGQGTLVTVSS

SEQ ID NO: 41 - protein, 117 aa, synthetic construct
QLQLQESGPGLVKPSETLSLTCTVSGYSITSDYAWNWIRQPPGKGLEWMGYISYSGSISYNPSLKSRITISRDTSKNQFSLKLSSVTAADTAVYYCARSRYSWFDYWGQGTLVTVSS

SEQ ID NO: 42 - protein, 117 aa, synthetic construct
QVQLQESGPGLVKPSETLSLTCTVSGYSITSDYAWNWIRQFPGKGLEWMGYISYSGSISYNPSLKSRITISRDTSKNQFFLKLSSVTAADTAVYYCARSRYSWFDYWGQGTLVTVSS

SEQ ID NO: 43 - protein, 117 aa, synthetic construct
QVQLQESGPGLVKPSETLSLTCTVTGYSITSDYAWNWIRQFPGKKLEWMGYISYSGSISYNPSLKSRITISRDTSKNQFFLQLSSVTAADTAVYYCARSRYSWFDYWGQGTLVTVSS

SEQ ID NO: 44 - protein, 111 aa, synthetic construct
EIVLTQSPGTLSLSPGERATLSCSASESVDTYGNSFMHWYQQKPGQAPRLLIYRASSLESGIPDRFSGSGSGTDFTLTISRLEPEDFAVYYCQQSNEDPYTFGGGTKVEIK

SEQ ID NO: 45 - protein, 111 aa, synthetic construct
EIVLTQSPGTLSLSPGERATLSCSASESVDTYGNSFMHWYQQKPGQPPRLLIYRASSLESGIPVRFSGSGSRTDFTLTISRLEPEDFAVYYCQQSNEDPYTFGGGTKVEIK

SEQ ID NO: 46 - protein, 111 aa, synthetic construct
EIVLTQSPGTLSLSPGERATLSCSASESVDTYGNSFMHWYQQKPGQPPRLLIYRASSLESGIPVRFSGSGSRTDFTLTISRVEAEDFAVYYCQQSNEDPYTFGGGTKLEIK

SEQ ID NO: 47 - protein, 111 aa, synthetic construct
DIVLTQSPGTLSVSPGERATLSCSASESVDTYGNSFMHWYQQKPGQPPKLLIYRASSLESGIPVRFSGSGSRTDFTLTISRVEAEDFAVYYCQQSNEDPYTFGGGTKLEIK

SEQ ID NO: 48 - protein, 117 aa, synthetic construct
QLQLQESGPGLVKPSETLSLTCTVSGYSITSDFAWNWIRQPPGKGLEWIGYISFSGSTSYNPSLKSRVTISVDTSKNQFSLKLSSVTAADTAVYYCARSRYSWFAYWGQGTLVTVSS

SEQ ID NO: 49 - protein, 117 aa, synthetic construct
QLQLQESGPGLVKPSETLSLTCTVSGYSITSDFAWNWIRQPPGKGLEWMGYISFSGSTSYNPSLKSRVTISRDTSKNQFSLKLSSVTAADTAVYYCAGSRYSWFAYWGQGTLVTVSS

SEQ ID NO: 50 - protein, 117 aa, synthetic construct
QVQLQESGPGLVKPSETLSLTCTVSGYSITSDFAWNWIRQFPGKGLEWMGYISFSGSTSYNPSLKSRITISRDTSKNQFSLKLSSVTAADTAVYYCAGSRYSWFAYWGQGTLVTVSS

SEQ ID NO: 51 - protein, 117 aa, synthetic construct
QVQLQESGPGLVKPSETLSLTCTVSGYSITSDFAWNWIRQFPGKGLEWMGYISFSGSTSYNPSLKSRITISRDTSKNQFFLQLSSVTAEDTAVYYCAGSRYSWFAYWGQGTLVTVSS

SEQ ID NO: 52 - protein, 111 aa, synthetic construct
DIQMTQSPSSLSASVGDRVTITCRASESVDTYGNSFMHWYQQKPGKAPKLLIYRASNLESGVPSRFSGSGSGTDFTLTISSLQPEDFATYYCQQSNEDPYTFGGGTKVEIK

SEQ ID NO: 53 - protein, 111 aa, synthetic construct
DIQLTQSPSSLSASVGDRVTITCRASESVDTYGNSFMHWYQQKPGKPPKLLIYRASNLESGIPSRFSGSGSGTDFTLTISSLQPEDFATYYCQQSNEDPYTFGGGTKVEIK

SEQ ID NO: 54 - protein, 111 aa, synthetic construct
DIVLTQSPSSLSASVGDRVTITCRASESVDTYGNSFMHWYQQKPGQPPKLLIYRASNLESGIPARFSGSGSGTDFTLTISSVQPEDFATYYCQQSNEDPYTFGGGTKVEIK

SEQ ID NO: 55 - protein, 111 aa, synthetic construct
DIVLTQSPSSLSVSVGDRATITCRASESVDTYGNSFMHWYQQKPGQPPKLLIYRASNLESGIPARFSGSGSRTDFTLTISSVQPEDFATYYCQQSNEDPYTFGGGTKLEIK

SEQ ID NO: 56 - protein, 113 aa, synthetic construct
QVQLVQSGAEVKKPGASVKVSCKASGYTFTDYWVSWVRQAPGQGLEWMGEIYPKNGVSNFNEKFKGRVTMTTDTSTSTAYMELRSLRSDDTAVYYCARQRDYWGQGTLVTVSS

SEQ ID NO: 57 - protein, 113 aa, synthetic construct
QVQLVQSGAEVVKPGASVKVSCKASGYTFTDYWVSWVRQAPGQGLEWIGEIYPKNGVSNFNEKFKGRVTMTVDTSTSTAYMELRSLRSDDTAVYYCTRQRDYWGQGTLVTVSS

SEQ ID NO: 58 - protein, 113 aa, synthetic construct
QVQLVQSGAEVKVPGASVKMSCKASGYTFTDYWVSWVRQSPGQGLEWIGEIYPKNGVSNFNEKFKGRVTLTVDKSTSTAYMELRSLRSDDTAVYYCTRQRDYWGQGVLVTVSS

SEQ ID NO: 59 - protein, 113 aa, synthetic construct
QVQLVQSGAEVVKPGASVKMSCKASGYTFTDYWVSWVKQSPGQGLEWIGEIYPKNGVSNFNEKFKGKATLTVDKSTSTAYMELRRLRSDDTAVFYCTRQRDYWGQGVLVTVSS

SEQ ID NO: 60 - protein, 112 aa, synthetic construct
DVVMTQSPLSLPVTLGQPASISCRSSQSLEYGDGYTYLEWFQQRPGQSPRRLIYGVSNRFSGVPDRFSGSGSGTDFTLKISRVEAEDVGVYYCFQATHDPNTFGQGTKLEIK

SEQ ID NO: 61 - protein, 112 aa, synthetic construct
DVVMTQSPLSLPVTLGQPASISCRSSQSLEYGDGYTYLEWYLQRPGQSPRLLIYGVSNRFSGVPDRFSGSGSGTDFTLKISRVEAEDVGVYYCFQATHDPNTFGQGTKLEIK

SEQ ID NO: 62 - protein, 112 aa, synthetic construct
DVVMTQSPLSLPVTLGQPASISCRSSQSLEYGDGYTYLEWYLQRPGQSPRLLIYGVSNRFSGVPDRFIGSGSGTDFTLKISRVEAEDVGVYYCFQATHDPNTFGAGTKLEIK

SEQ ID NO: 63 - protein, 112 aa, synthetic construct
DVVLTQSPLSLPVTLGQPASISCRSSQSLEYGDGYTYLEWYLQRPGQSPQLLIYGVSNRFSGVPDRFIGSGSGTDFTLKISRVEPEDVGVYYCFQATHDPNTFGAGTKLEIK

SEQ ID NO: 64 - protein, 113 aa, synthetic construct
QVQLVQSGAEVKKPGASVKVSCKASGYTFTDYWVSWVRQAPGQGLEWMGEIYPKEGVSNFNEKFKGRVTMTTDTSTSTAYMELRSLRSDDTAVYYCARQRDYWGQGTLVTVSS

SEQ ID NO: 65 - protein, 113 aa, synthetic construct
QVQLVQSGAEVKKPGASVKVSCKASGYTFTDYWVSWVRQAPGQGLEWMGEIYPKNDVSNFNEKFKGRVTMTTDTSTSTAYMELRSLRSDDTAVYYCARQRDYWGQGTLVTVSS

SEQ ID NO: 66 - protein, 113 aa, synthetic construct
QVQLVQSGAEVKKPGASVKVSCKASGYTFTDYWVSWVRQAPGQGLEWMGEIYPKNQVSNFNEKFKGRVTMTTDTSTSTAYMELRSLRSDDTAVYYCARQRDYWGQGTLVTVSS

SEQ ID NO: 67 - protein, 112 aa, synthetic construct
DVVMTQSPLSLPVTLGQPASISCRSSQSLEYGSGYTYLEWYLQRPGQSPRLLIYGVSNRFSGVPDRFSGSGSGTDFTLKISRVEAEDVGVYYCFQATHDPNTFGQGTKLEIK

SEQ ID NO: 68 - protein, 112 aa, synthetic construct
DVVMTQSPLSLPVTLGQPASISCRSSQSLEYGDRYTYLEWYLQRPGQSPRLLIYGVSNRFSGVPDRFSGSGSGTDFTLKISRVEAEDVGVYYCFQATHDPNTFGQGTKLEIK

SEQ ID NO: 69 - protein, 112 aa, synthetic construct
DVVMTQSPLSLPVTLGQPASISCRSSQSLEYGTGYTYLEWYLQRPGQSPRLLIYGVSNRFSGVPDRFSGSGSGTDFTLKISRVEAEDVGVYYCFQATHDPNTFGQGTKLEIK

SEQ ID NO: 70 - protein, 6 aa, synthetic construct
  VARIANT 3: note = X is F or Y
SDXAWN

SEQ ID NO: 71 - protein, 16 aa, synthetic construct
  VARIANT 3: note = X is N or S
  VARIANT 4: note = X is F or Y
  VARIANT 8: note = X is I or T
YIXXSGSXSYNPSLKS

SEQ ID NO: 72 - protein, 8 aa, synthetic construct
  VARIANT 7: note = X is A or D
SRYSWFXY

SEQ ID NO: 73 - protein, 15 aa, synthetic construct
  VARIANT 1: note = X is R or S
XASESVDTYGNSFMH

SEQ ID NO: 74 - protein, 7 aa, synthetic construct
  VARIANT 4: note = X is N or S
RASXLES

SEQ ID NO: 75 - protein, 17 aa, synthetic construct
  VARIANT 5: note = X is E or K
  VARIANT 6: note = X is E or N
  VARIANT 7: note = X is D, G or Q
  VARIANT 8: note = X is I or V
  VARIANT 9: note = X is S or T
EIYPXXXXXNFNEKFKG

SEQ ID NO: 76 - protein, 16 aa, synthetic construct
  VARIANT 7: note = X is E or K
  VARIANT 10: note = X is D, S or T
  VARIANT 11: note = X is G or R
RSSQSLXYGXXYTYLE